(19) United States
(12) Patent Application Publication
(10) Pub. No.: US 2006/0172014 A1
(43) Pub. Date: Aug. 3, 2006

(54) Treatment of lung cancer with active vitamin D compounds in combination with other treatments

(75) Inventors: John G. Curd, Hillsborough, CA (US); Martha J. Whitehouse, San Francisco, CA (US); Jeffrey L. Cleland, San Carlos, CA (US)

Correspondence Address: STERNE, KESSLER, GOLDSTEIN & FOX PLLC, 1100 NEW YORK AVENUE, N.W., WASHINGTON, DC, 20005, US

(73) Assignee: Novacea, Inc.

(21) Appl. No.: 11/298,927

(22) Filed: Dec. 12, 2005

Related U.S. Application Data
(63) Continuation-in-part of application No. PCT/US04/18427, filed on Jun. 10, 2004.
(60) Provisional application No. 60/477,339, filed on Jun. 11, 2003.
(60) Provisional application No. 60/569,245, filed on May 10, 2004.

(51) Int. Cl. A61K 33/24; A61K 31/7072; A61K 31/7048; A61K 31/704; A61K 31/4745; A61K 31/59; A61K 31/66; A61K 31/337

(52) U.S. Cl. 424/649; 514/34; 514/27; 514/167; 514/49; 514/109; 514/283; 514/492; 514/449

(57) ABSTRACT

The present invention relates to a method for treating or ameliorating lung cancer in an animal by administering to the animal active vitamin D compounds or mimics thereof by high dose pulse administration in combination with one or more chemotherapeutic agents or radiotherapeutic agents/treatments.

### BACKGROUND OF THE INVENTION

1. Field of the Invention

The present invention relates to a method for treating or ameliorating lung cancer in an animal by administering to the animal active vitamin D compounds or mimics thereof by high dose pulse administration in combination with one or more chemotherapeutic agents or radiotherapeutic agents/treatments.

2. Related Art

Lung cancer is the leading cause of death due to cancer in the United States. The American Cancer Society estimates that 172,570 new cases of lung cancer will be diagnosed in the United States in 2003 and that there will be 163,510 deaths due to this disease. American Cancer Society, “Cancer Facts and Figures 2005,” 2005, Atlanta, p. 5. The prognosis for patients with lung cancer remains poor. Although improved surgical techniques have led to an increase in one-year survival for lung cancer from 34% in 1975 to 42% in 1998, the five-year survival rate for all stages combined is only 15%. This poor prognosis is primarily due to the fact that only a small portion of cases are diagnosed at an early stage.

Non-small cell lung cancer (NSCLC) is the predominant form of lung cancer, accounting for about 80% of all cases. When localized, NSCLC can be treated surgically or in some cases with combined radiation and chemotherapy. However, about 50% of surgically resectable cases and about 80% of locally advanced cases will relapse.

Therapy of locally advanced (Stage IIIB) NSCLC or NSCLC with distant metastases (Stage IV) is not curative. Radiation therapy can be used for palliation of symptoms either at the primary site or at sites of distant metastases.

Chemotherapy for NSCLC, while not curative, has been shown to prolong survival in patients with unresectable disease. Souquet et al., Lancet 342:19-21 (1993). A number of different chemotherapy agents have been shown to have single-agent activity in NSCLC. These include cisplatin, carboplatin, vinorelbine, gemcitabine, paclitaxel, and docetaxel. For first line therapy of Stage IIIB or Stage IV NSCLC, these agents are generally used in combination. In most cases, first-line chemotherapy consists of a platinum-based agent, either cisplatin or carboplatin, and another chemotherapeutic agent. In a study recently reported by Schiller and coworkers, four platinum-based chemotherapeutic agents were compared and found to produce equivalent objective tumor response rates and overall survival. Schiller et al., N. Eng. J Med. 346:92-8 (2002). The response rate in this study was 19% and the median survival was 7.9 months. More recently, docetaxel (TAXOTERE) has been approved in combination with cisplatin as first-line therapy for NSCLC. Only one agent, docetaxel, has been approved in the United States by the Food and Drug Administration for use as a second-line chemotherapy in NSCLC.

Vitamin D is a fat soluble vitamin which is essential as a positive regulator of calcium homeostasis. (See Harrison's Principles of Internal Medicine: Part Thirteen, “Disorders of Bone and Mineral Metabolism,” Chapter 353, pp. 2214-2226, A. S. Fauci et al., (eds.), McGraw-Hill, New York (1998)). The active form of vitamin D is 1α,25-dihydroxyvitamin D₃, also known as calcitriol. Specific nuclear receptors for active vitamin D compounds have been discovered in cells from diverse organs not involved in calcium homeostasis. Miller et al., Cancer Res. 52:515-520 (1992). In addition to influencing calcium homeostasis, active vitamin D compounds have been implicated in osteogenesis, modulation of immune response, modulation of the process of insulin secretion by pancreatic B cells, muscle cell function, and the differentiation and growth of epidermal and hematopoietic tissues.

Moreover, there have been many reports demonstrating the utility of active vitamin D compounds in the treatment of hyperproliferative diseases,. (e.g., cancer, psoriasis). For example, it has been shown that certain vitamin D compounds and analogues possess potent antileukemic activity by virtue of inducing the differentiation of malignant cells (specifically, leukemic cells) to non-malignant macrophages (monocytes) and are useful in the treatment of leukemia. Suda et al., U.S. Pat. No. 4,391,802; Partridge et al., U.S. Pat. No. 4,594,340. Anti-proliferative and differentiating actions of calcitriol and other vitamin D₃ analogues have also been reported with respect to the treatment of prostate cancer (Bishop et al., U.S. Pat. No. 5,795,882), skin cancer (Chida et al., Cancer Research 45:5426-5430 (1985)), and colon cancer (Disman et al., Cancer Res. 47:21-25 (1987)). Vitamin D has been shown to inhibit the metastasis of lung cancer cells implanted in animals. Sato et al., Tohoku J. Exp. Med. 138:445-446 (1982). Calcitriol was found to inhibit the growth of a lung cancer cell line containing high levels of vitamin D receptor but not lung cancer cell lines having insignificant levels of the receptor. Higashimoto et al., Anticancer Res. 16:2653-2660 (1996). Other reports suggesting important therapeutic uses of active vitamin D compounds are summarized in Rodriguez et al., U.S. Pat. No. 6,034,074.

Active vitamin D compounds have also been administered in combination with other pharmaceutical agents, in particular cytotoxic agents for the treatment of hyperproliferative disease. For example, it has been shown that pretreatment of hyperproliferative cells with active vitamin D compounds followed by treatment with cytotoxic agents enhances the efficacy of the cytotoxic agents (U.S. Pat. Nos. 6,087,350; 6,559,139).

Although the administration of active vitamin D compounds may result in substantial therapeutic benefits, the treatment of hyperproliferative diseases with such compounds is limited by the effects these compounds have on calcium metabolism. At the levels required in vivo for effective use as anti-proliferative agents, active vitamin D compounds can induce markedly elevated and potentially dangerous blood calcium levels by virtue of their inherent calcemic activity. That is, the clinical use of calcitriol and other active vitamin D compounds as anti-proliferative agents is severely limited by the risk of hypercalcemia.

A great deal of research has gone into the identification of vitamin D analogs and derivatives that maintain an anti-proliferative effect but have a decreased effect on calcium metabolism. Hundreds of compounds have been created, many with reduced hypercalcemic effects, but no compounds have been discovered that maintain anti-proliferative activity while completely eliminating the hypercalcemic effect.

It has been shown that the problem of systemic hypercalcemia can be overcome by “high dose pulse administration” (HDPA) of a sufficient dose of an active vitamin D compound such that an anti-proliferative effect is observed while avoiding the development of severe hypercalcemia. According to U.S. Pat. No. 6,521,608, the active vitamin D compound may be administered no more than every three days, for example, once a week at a dose of at least 0.12 μg/kg per day (8.4 μg in a 70 kg person). Pharmaceutical compositions used in the HDPA regimen of U.S. Pat. No. 6,521,608 comprise 5-100 μg of active vitamin D compound and may be administered in the form for oral, intravenous, intramuscular, topical, transdermal, sublingual, intranasal, intratumoral or other preparations. In a Phase I trial of weekly administration of calcitriol to patients with refractory malignancies, HDPA of calcitriol was shown to produce no dose-limiting toxicity and to produce mean peak calcitriol levels within the therapeutic range. Beer et al., Cancer 91:2431-39 (2001).

### SUMMARY OF THE INVENTION

One aspect of the present invention is a method for treating or ameliorating lung cancer in an animal comprising administering to the animal a therapeutically effective amount of an active vitamin D compound or a mimic thereof by HDPA in combination with one or more chemotherapeutic agents or radiotherapeutic agents/treatments. In another preferred aspect of the invention, the active vitamin D compound or a mimic thereof has a reduced hypercalcemic effect, allowing higher doses of the compound to be administered to an animal without inducing hypercalcemia.

In preferred embodiments of the invention, the one or more chemotherapeutic agents can be ones that have been demonstrated to be effective in the treatment or amelioration of lung cancer, either alone or in combination therapy.

In preferred embodiments of the invention, the one or more radiotherapeutic agents or treatments can be external-beam radiation therapy, brachytherapy, thermotherapy, radiosurgery, charged-particle radiotherapy, neutron radiotherapy, photodynamic therapy, or radionuclide therapy.

In one embodiment of the invention, the active vitamin D compound or a mimic thereof can be administered prior to, during, and/or beyond administration of the one or more chemotherapeutic agents or radiotherapeutic agents or treatments. In another embodiment of the invention, the method of administering an active vitamin D compound in combination with one or more chemotherapeutic agents or radiotherapeutic agents or treatments is repeated more than once.

The combination of an active vitamin D compound or a mimic thereof and one or more chemotherapeutic agents or radiotherapeutic agents or treatments of the present invention can have additive potency or an additive therapeutic effect. The invention also encompasses synergistic combinations where the therapeutic efficacy is greater than additive. Preferably, such combinations also reduce or avoid unwanted or adverse effects. In certain embodiments, the combination therapies encompassed by the invention provide an improved overall therapy relative to administration of an active vitamin D compound or any chemotherapeutic agent or radiotherapeutic agent or treatment alone. In certain embodiments, doses of existing or experimental chemotherapeutic agents or radiotherapeutic agents or treatments can be reduced or administered less frequently which increases patient compliance, thereby improving therapy and reducing unwanted or adverse effects.

Further, the methods of the invention are useful not only with previously untreated patients but also useful in the treatment of patients partially or completely refractory to current standard and/or experimental cancer therapies, including but not limited to radiotherapies, chemotherapies, and/or surgery. In a preferred embodiment, the invention provides therapeutic methods for the treatment or amelioration of a lung cancer that has been shown to be or may be refractory or non-responsive to other therapies.

### DETAILED DESCRIPTION OF THE INVENTION

One aspect of the present invention is a method for treating or ameliorating lung cancer in an animal comprising administering to the animal an a therapeutically effective amount of active vitamin D compound or a mimic thereof by HDPA in combination with one or more chemotherapeutic agents or radiotherapeutic agents/treatments, which agents or treatments are currently being used, have been used, or are known to be useful in the treatment or amelioration of lung cancer. In another preferred aspect of the invention, the active vitamin D compound or a mimic thereof has a reduced hypercalcemic effect, allowing higher doses of the compound to be administered to an animal without inducing hypercalcemia.

While not intending to be bound by any specific theory, it is believed that there are two distinct, possibly interrelated molecular mechanisms that could underlie the ability of vitamin D compounds to act in an additive or synergistic fashion with chemotherapeutic agents or radiotherapeutic agents or treatments in the treatment of lung cancer. One mechanism is the ability of active vitamin D compounds to arrest cells in the G₀/G₁ phase of the cell cycle, probably through the inhibition of cell cycle dependent kinases and the modulation of the regulators of these kinases. The second mechanism is the ability of active vitamin D compounds to modulate several key regulatory molecules that control apoptosis (e.g., bcl-2, IAPs, Bax) to create a significantly enhanced potential for apoptosis in the cells (proapoptotic changes). Following exposure to active vitamin D compounds, cells are more sensitive to induction of apoptosis by chemotherapeutic agents or radiotherapeutic agents and treatments.

As used herein, the term “therapeutically effective amount” refers to that amount of the therapeutic agent sufficient to result in amelioration of one or more symptoms of a disorder, or prevent advancement of a disorder, or cause regression of the disorder. For example, with respect to the treatment of lung cancer, a therapeutically effective amount preferably refers to the amount of a therapeutic agent that decreases the rate of tumor growth, decreases tumor mass, decreases the number of metastases, increases time to tumor progression, or increases survival time by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The term “an active vitamin D compound in combination with one or more chemotherapeutic agents or radiotherapeutic agents or treatments,” as used herein, is intended to refer to the combined administration of an active vitamin D compound or a mimic thereof and one or more chemotherapeutic agents or radiotherapeutic agents or treatments, wherein the active vitamin D compound or a mimic thereof can be administered prior to, concurrently with, or after the administration of the chemotherapeutic agents or radiotherapeutic agents or treatments. The active vitamin D compound or a mimic thereof can be administered up to three months prior to or after the chemotherapeutic agents or radiotherapeutic agents or treatments and still be considered to be a combination treatment.

The term “lung cancer,” as used herein, is intended to refer to any known lung cancer, and may include, but is not limited to, non-small cell lung cancer, squamous cell carcinoma, epidermoid carcinoma, adenocarcinoma, bronchoalveolar cell carcinoma, small cell lung cancer, oat cell carcinoma, large-cell carcinoma, giant cell carcinoma, clear cell carcinoma, adenosquamous carcinoma, carcinoid tumor, bronchial gland carcinoma, soft-tissue tumor, and mesothelial tumor. See Fishman et al., 1985, Medicine, 2d Ed., J. B. Lippincott Co., Philadelphia, Pa. and Murphy et al., 1997, Informed Decisions: The Complete Book of Cancer Diagnosis, Treatment, and Recovery, Viking Penguin, New York, N.Y., for a review of such disorders.

The term “active vitamin D compound,” as used herein, is intended to refer to a vitamin D compound that is or becomes biologically active (e.g., binds to and stimulates the vitamin D receptor) when administered to a subject or contacted with cells. Active vitamin D compounds include compounds that cause hypercalcemia and compounds that do not cause hypercalcemia upon administration. The biological activity of the compound may be manifested or increased following metabolism of the compound after administration to a subject. The biological activity of a vitamin D compound can be assessed by assays well known to one of skill in the art such as, e.g., immunoassays that measure the expression of a specific gene regulated by vitamin D. Vitamin D compounds exist in several forms with different levels of activity in the body. For example, a vitamin D compound may be partially activated by first undergoing hydroxylation in the liver at the carbon-25 position and then may be fully activated in the kidney by further hydroxylation at the carbon-1 position. The prototypical active vitamin D compound is 1α,25-hydroxyvitamin D₃, also known as calcitriol. A large number of other active vitamin D compounds are known and can be used in the practice of the invention. The active vitamin D compounds of the present invention include but are not limited to analogs, homologs, mimics, and derivatives of vitamin D compounds such as those described in the following patents, each of which is incorporated by reference: U.S. Pat. No. 4,391,802 (1α-hydroxyvitamin D derivatives); U.S. Pat. No. 4,717,721 (1α-hydroxy derivatives with a 17 side chain greater in length than the cholesterol or ergosterol side chains); U.S. Pat. No. 4,851,401 (cyclopentano-vitamin D analogs); U.S. Pat. Nos. 4,866,048 and 5,145,846 (vitamin D₃ analogues with alkynyl, alkenyl, and alkanyl side chains); U.S. Pat. No. 5,120,722 (trihydroxycalciferol); U.S. Pat. No. 5,547,947 (fluoro-cholecalciferol compounds); U.S. Pat. No. 5,446,035 (methyl substituted vitamin D); U.S. Pat. No. 5,411,949 (23-oxa-derivatives); U.S. Pat. No. 5,237,110 (19-nor-vitamin D compounds; U.S. Pat. No. 4,857,518 (hydroxylated 24-homo-vitamin D derivatives). Particular examples include ROCALTROL (Roche Laboratories); CALCIJEX injectable calcitriol; investigational drugs from Leo Pharmaceuticals including EB 1089 (24a,26a,27a-trihomo-22,24-diene-1α,25-(OH)₂-D₃, KH 1060 (20-epi-22-oxa-24a,26a,27a-trihomo-1α,25-(OH)₂-D₃), MC 1288 (1,25-(OH)₂-20-epi-D₃) and MC 903 (calcipotriol, 1α24s-(OH)₂-22-ene-26,27-dehydro-D₃); Roche Pharmaceutical drugs that include 1,25-(OH)₂-16-ene-D₃, 1,25-(OH)₂-16-ene-23-yne-D₃, and 25-(OH)₂-16-ene-23-yne-D₃; Chugai Pharmaceuticals 22-oxacalcitriol (22-oxa-1α,25-(OH)₂-D₃; 1α-(OH)-D₅ from the University of Illinois; and drugs from the Institute of Medical Chemistry-Schering AG that include ZK 161422 (20-methyl-1,25-(OH)₂-D₃) and ZK 157202 (20-methyl-23-ene-1,25-(OH)₂-D₃); 1α-(OH)-D₂; 1α-(OH)-D₃, 1α-(OH)-D₄, 25-(OH)-D₂; 25-(OH)-D₃; and 25-(OH)-D₄. Additional examples include 1α,25-(OH)₂-26,27-d₆-D₃; 1α,25-(OH)₂-22-ene-D₃; 1α,25-(OH)₂-D₃; 1α,25-(OH)₂-D₂; 1α,25-(OH)₂-D₄; 1α,24,25-(OH)₃-D₃; 1α,24,25-(OH)₃-D₂; 1α,24,25-(OH)₃-D₄; 1α-(OH)-25-FD₃; 1α-(OH)-25-FD₄; 1α-(OH)-25-FD₂; 1α,24-(OH)₂-D₄; 1α,24-(OH)₂-D₃; 1α,24-(OH)₂-D₂; 1α,24-(OH)₂-25-FD₄; 1α,24-(OH)₂-25-FD₃; 1α,24-(OH)₂-25-FD₂; 1α,25-(OH)₂-26,27-F₆-22-ene-D₃; 1α,25-(OH)₂-26,27-F₆-D₃; 1α,25 S-(OH)₂-26-F₃-D₃; 1α,25-(OH)₂-24-F₂-D₃; 1α25S,26-(OH)₂-22-ene-D₃; 1α,25R,26-(OH)₂-22-ene-D₃; 1α,25-(OH)₂-D₂; 1α,25-(OH)₂-24-epi-D₃; 1α,25-(OH)₂-23-yne-D₃; 1α,25-(OH)₂-24R—F-D₃; 1α,25S,26-(OH)₂-D₃; 1α,24R-(OH)₂-25F-D₃; 1α,25-(OH)₂-26,27-F₆-23-yne-D₃; 1α,25R-(OH)₂-26-F₃-D₃; 1α,25,28-(OH)₃-D₂; 1α25-(OH)₂-16-ene-23-yne-D₃; 1α,24R,25-(OH)₃-D₃; 1α,25-(OH)₂-26,27-F₆-23-ene-D₃; 1α,25R-(OH)₂-22-ene-26-F₃-D₃; 1α,25S-(OH)₂-22-ene-26-F₃-D₃; 1α,25R-(OH)₂-D₃-26,26,26-d₃; 1α,25S-(OH)₂-D₃-26,26,26-d₃; and 1α,25R-(OH)₂-22-ene-D₃-26,26,26-d₃. Additional examples can be found in U.S. Pat. No. 6,521,608. See also, e.g., U.S. Pat. Nos. 6,503,893, 6,482,812, 6,441,207, 6,410,523, 6,399,797, 6,392,071, 6,376,480, 6,372,926, 6,372,731, 6,359,152, 6,329,357, 6,326,503, 6,310,226, 6,288,249, 6,281,249, 6,277,837, 6,218,430, 6,207,656, 6,197,982, 6,127,559, 6,103,709, 6,080,878, 6,075,015, 6,072,062, 6,043,385, 6,017,908, 6,017,907, 6,013,814, 5,994,332, 5,976,784, 5,972,917, 5,945,410, 5,939,406, 5,936,105, 5,932,565, 5,929,056, 5,919,986, 5,905,074, 5,883,271, 5,880,113, 5,877,168, 5,872,140, 5,847,173, 5,843,927, 5,840,938, 5,830,885, 5,824,811, 5,811,562, 5,786,347, 5,767,111, 5,756,733, 5,716,945, 5,710,142, 5,700,791, 5,665,716, 5,663,157, 5,637,742, 5,612,325, 5,589,471, 5,585,368, 5,583,125, 5,565,589, 5,565,442, 5,554,599, 5,545,633, 5,532,228, 5,508,392, 5,508,274, 5,478,955, 5,457,217, 5,447,924, 5,446,034, 5,414,098, 5,403,940, 5,384,313, 5,374,629, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,804,502, 4,857,518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,367,177, 4,336,193, 4,360,472, 4,360,471, 4,307,231, 4,307,025, 4,358,406, 4,305,880, 4,279,826, and 4,248,791.

The term “mimic” as used herein is intended to refer to non-secosteroidal vitamin D mimic compounds. In general, these non-secosteroidal vitamin D mimics are compounds that do not structurally fall within the class of compounds generally known as vitamin D compounds but which modulate the activity of vitamin D nuclear receptors. Examples of such vitamin D mimics include bis-aryl derivatives disclosed by U.S. Pat. No. 6,218,430 and WO publication 2005/037755. Additional examples of non-secosteroidal vitamin D mimic compounds suitable for the present invention can be found in U.S. Pat. Nos. 6,831,106; 6,706,725; 6,689,922; 6,548,715; 6,288,249; 6,184,422, 6,017,907, 6,858,595 and 6,358,939.

In one aspect the invention is drawn to methods employing non-secosteroidal vitamin D mimic compounds having Formula I:

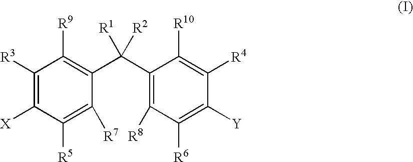

wherein:

R¹ and R² are each independently halo, haloalkyl, pseudohalo, optionally substituted alkyl, optionally substituted alkenyl, optionally substituted alkynyl, optionally substituted cycloalkyl, optionally substituted heterocyclyl, optionally substituted aryl or optionally substituted heteroaryl; or

R¹ and R², together with the carbon atom to which they are attached, form an optionally substituted cycloalkyl consisting of:

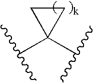

wherein k is an integer from 1 to 6; or

R¹ and R², together with the carbon atom to which they are attached, form an optionally substituted heterocyclyl selected from a group consisting of:

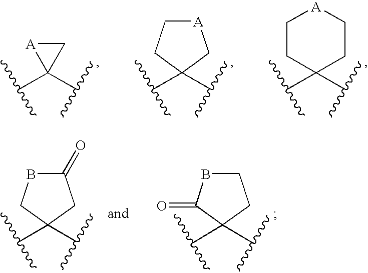

wherein A is —O—, —NR^{x}—, —S—, —S(O)— or —S(O)₂— wherein R^{x} is hydrogen, alkyl, haloalkyl, cycloalkyl, heterocyclyl, aryl, heteroaryl, —R¹⁴—C(J)R¹⁵, —R¹⁴—C(J)OR¹⁵, —R¹⁴—C(J)R¹⁶OR¹⁵, —R¹⁴—C(J)SR¹⁶, —R¹⁴—C(J)N(R¹⁸)R¹⁹, —R¹⁴—C(J)N(R¹⁷)N(R¹⁸)R¹⁹, —R¹⁴—C(J)N(R¹⁷)S(O)ₚR²⁰, —R¹⁴—S(O)ₚN(R¹⁸)R¹⁹, or —R¹⁴—S(O)ₚR²⁰; and wherein B is —O—, —S— or —NR^{y} where R^{y} is hydrogen, alkyl, haloalkyl, aryl or heteroaryl; and wherein each p is independently 0 to 2;

R³ and R⁴ are each independently hydrogen, alkyl, alkenyl, alkynyl, cycloalkyl, heterocyclyl, aryl, heteroaryl, halo, pseudohalo, nitro, cyano, azido, —R¹⁴—OR , —R¹⁴—N(R¹⁸)R¹⁹, —R¹⁴—SR¹⁵, —R¹⁴—OC(J)R¹⁵, —R¹⁴—NR¹⁷C(J)R¹⁵, —R¹⁴—OC(J)N(R¹⁸)R¹⁹, —R¹⁴—NR¹⁷C(J)N(R¹⁸)R¹⁹, —R¹⁴—NR¹⁷C(J)OR¹⁵, —R¹⁴—C(J)R¹⁵—R¹⁴—C(J)OR¹⁵, —R¹⁴—C(J)SR¹⁵, —R¹⁴—C(J)N(R¹⁸)R¹⁹, or —R¹⁴—C(J)N(R¹⁷)N(R¹⁸)R¹⁹;

R⁵, R⁶, R⁷, R⁸, R⁹, R¹⁰ are each independently hydrogen, halo, hydroxy, amino, pseudohalo, cyano, nitro, alkyl, haloalkyl, alkoxy or haloalkoxy;

X is R²⁵;

Y is independently R³⁰, —OR³¹, —SR³² or —N(R³³)(R³⁴);

R²⁵ and R³⁰ are each independently selected from (i) or (ii) as follows:

(i) optionally substituted alkyl that may be substituted with one to ten substituents each independently selected from a group consisting of halo, pseudohalo, nitro, cyano, thioxo, azido, amidino, guanidino, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —OR¹⁵, —OR¹⁶OR¹⁵, —N(R¹⁸)R¹⁹, —N(R¹⁷)N(R¹⁸)R¹⁹, —SR¹⁵, —SR¹⁶SR¹⁵, —N(R¹⁷)N(R¹⁷)S(O)ₚR²⁰, —OC(J)R¹⁵, —NR¹⁷C(J)R¹⁵, —OC(J)N(R¹⁸)R¹⁹, —NR¹⁷C(J)N(R¹⁸)R¹⁹, —NR¹⁷C(J)OR¹⁵, —OC(J)OR¹⁵, —P(R²¹)₂, —P(O)(R²¹)₂, —OP(O)(R²¹)₂, —C(J)R¹⁵, —C(J)OR¹⁵, —C(J)SR¹⁶, —C(J)(R¹⁸)R¹⁹, —C(J)N(R¹⁷)N(R¹⁸)R¹⁹, —C(J)N(R¹⁷)N(R¹⁷)S(O)ₚR²⁰, —C(R¹⁷)═NOR¹⁵, —C(R¹⁷)═NR¹⁷, —C(R¹⁷)═NN(R¹⁸)R¹⁹ and —C(═NR¹⁷)N(R¹⁸)R¹⁹; or

(ii) optionally substituted alkenyl or optionally substituted alkynyl, either of which may be substituted with one to ten substituents each independently selected from a group consisting of oxo, thioxo, halo, pseudohalo, nitro, cyano, azido, amidino, guanidino, —OR¹⁵, —OR¹⁶OR¹⁵, —N(R¹⁸)R¹⁹, —N(R¹⁷)N(R¹⁸)R¹⁹, —SR⁵, —SR¹⁶SR¹⁵, —S(O)ₚR²⁰, —N(R¹⁷)S(O)ₚR²⁰, N(R¹⁷)N(R¹⁷)S(O)ₚR²⁰, —OC(J)R¹⁵, —NR¹⁷C(J)R¹⁵, —OC(J)N(R¹⁸)R¹⁹, —NR¹⁷C(J)N(R¹⁸)R¹⁹, —NR¹⁷C(J)OR¹⁵, —OC(J)OR¹⁵, —P(R²¹)₂, —P(O)(R²¹)₂, —OP(O)(R²¹)₂, —C(J)R¹⁵, —C(J)OR¹⁵, —C(J)SR¹⁶, —C(J)N(R¹⁸)R¹⁹, —C(J)N(R¹⁷)N(R¹⁸)R¹⁹, —C(J)N(R¹⁷)S(O)ₚR²⁰, —C(J)N(R¹⁷)N(R¹⁷)S(O)ₚR²⁰, —C(R¹⁷)═NOR¹⁵, —C(R¹⁷)═NR¹⁷, —C(R¹⁷)═NN(R¹⁸)R¹⁹, —C(═NR¹⁷)N(R¹⁸)R¹⁹, alkyl, haloalkyl, cycloalkyl, heterocyclyl, aryl and heteroaryl;

R³¹, R³², R³³, and R³⁴ are each independently optionally substituted alkyl, optionally substituted alkenyl, optionally substituted alkynyl or optionally substituted cycloalkyl; all of which may be optionally substituted with one to ten substituents each independently selected from a group consisting of oxo, halo, pseudohalo, nitro cyano, azido, amidino, guanidino —OR¹⁵, —OR¹⁶OR¹⁵, —N(R¹⁸)R¹⁹, —N(R¹⁷)N(R¹⁸)R¹⁹, —SR¹⁵, —SR¹⁶SR¹⁵, —S(O)ₚR²⁰, —N(R¹⁷)S(O)ₚR²⁰, —N(R¹⁷)N(R¹⁷)S(O)ₚR²⁰, —OC(J)R¹⁵, —NR¹⁷C(J)R¹⁵, —OC(J)N(R¹⁸)R¹⁹, —NR¹⁷C(J)N(R¹⁸)R¹⁹, —NR¹⁷C(J)OR¹⁵, —OC(J)OR¹⁵, —P(R²¹)₂, —P(O)(R²¹)₂, —OP(O)(R²¹)₂, —C(J)R¹⁵, C(J)OR¹⁵, —C(J)SR¹⁶, C(J)N(R¹⁸)R¹⁹, —C(J)N(R¹⁷)N(R¹⁸)R¹⁹, —C(J)N(R¹⁷)S(O)ₚR²⁰, —C(J)N(R¹⁷)N(R¹⁷)S(O)ₚR²⁰, —C(R¹⁷)═NOR¹⁵, —C(R¹⁷)═NR¹⁷, —C(R¹⁷)═NN(R¹⁸)R¹⁹, —C(═NR¹⁷)N(R¹⁸)R¹⁹, alkyl, cycloalkyl, heterocyclyl, aryl and heteroaryl, and R³⁴ can additionally be hydrogen;

where each R¹⁴ is independently a direct bond or alkylene;

where each R¹⁵ and R¹⁷ is independently hydrogen, optionally substituted alkyl, optionally substituted alkenyl, optionally substituted alkynyl, optionally substituted cycloalkyl, optionally substituted heterocyclyl, optionally substituted aryl or optionally substituted heteroaryl, all of which, when substituted, are substituted with one to five substituents each independently selected from halo, cyano, hydroxy and amino;

where each R¹⁶ and R²⁰ is independently optionally substituted alkyl, optionally substituted alkenyl, optionally substituted alkynyl, optionally substituted cycloalkyl, optionally substituted heterocyclyl, optionally substituted aryl or optionally substituted heteroaryl, all of which, when substituted, are substituted with one to five substituents each independently selected from halo, hydroxy, alkoxy and amino; and

where each R¹⁸ and R¹⁹ is independently hydrogen, optionally substituted alkyl, optionally substituted alkenyl, optionally substituted alkynyl, optionally substituted cycloalkyl, optionally substituted heterocyclyl, optionally substituted aryl or optionally substituted heteroaryl, all of which, when substituted, are substituted with one to five substituents each independently selected from halo, hydroxy, alkoxy and amino;

or where R¹⁸ and R¹⁹, together with the nitrogen atom to which they are attached, form a heterocyclyl or heteroaryl;

each R²¹ is independently alkyl, —OR²² or —N(R²³)R²⁴;

R²² is hydrogen, alkyl, haloalkyl, alkenyl, alkynyl, cycloalkyl, heterocyclyl, aryl, heteroaryl or aralkyl;

R²³ and R²⁴ are each independently hydrogen, alkyl, haloalkyl, alkenyl, alkynyl or cycloalkyl;

or R²³ and R²⁴, together with the nitrogen atom to which they are attached, form a heterocyclyl or heteroaryl;

each J is independently O or S;

as a single isomer, a mixture of isomers, or as a racemic mixture of isomers; as a solvate or polymorph; or as a prodrug or metabolite; or as a pharmaceutically acceptable salt thereof.

In one embodiment, R¹ and R² may form a substituted cyclohexyl, said cyclohexyl, when substituted at the 4-position relative to the gem-diaryl substituents, may be substituted with a substituent selected from the group consisting of halo, cyano, optionally substituted alkyl, optionally substituted alkenyl, optionally substituted alkynyl, optionally substituted aryl and optionally substituted heteroaryl.

In another embodiment, R²⁵ and R³⁰ are not —CH₂COOH; —CH₂-5-tetrazolyl; —CH₂COOMe; —CH₂COOEt; —CH₂NH(CH₂COOH); —CH₂N(C(O)Me)(CH₂COOH); —CH₂—N-pyrrolidin-2-one; —CH₂-(1-methylpyrrolidin-2-one-3-yl); —CH₂C(O)NH₂; —CH₂C(O)NMe₂; —CH₂C(O)NHMe; —CH₂C(O)—N-pyrrolidone; —CH(OH)COOH; —CH(OH)C(O)NH₂; —CH(OH)C(O)NHMe; —CH(OH)C(O)NMe₂; —CH(OH)C(O)NEt₂; —CH₂CH₂COOH; —CH₂CH₂COOMe; —CH₂CH₂COOEt; —CH₂CH₂C(O)NH₂; —CH₂CH₂C(O)NHMe; —CH₂CH₂C(O)NMe₂; or —CH₂CH₂-5-tetrazolyl.

In another aspect the invention is drawn to methods employing the following non-secosteroidal vitamin D mimic compounds:

3-(2-methyl-4-{2,2,2-trifluoro-1-[4-(2-hydroxy-3,3-dimethyl-butoxy)-3-methyl-phenyl]-1-phenyl-ethyl}-phenoxy)-propane-1,2-diol;

3-(4-{4-[4-(2-hydroxy-3,3-dimethyl-butoxy)-3-methyl-phenyl]-piperidin-4-yl}-2-methyl-phenoxy)-propane-1,2-diol;

3-(4-{4-[4-(2-hydroxy-3,3-dimethyl-butoxy)-3-methyl-phenyl]-piperidin-4-yl}-2-methyl-phenoxy)-propane-1,2(S)-diol;

1-{4-[4-(2(S),3-dihydroxy-propoxy)-3-methyl-phenyl]-4-[4-(2-hydroxy-3,3-dimethyl-butoxy)-3-methyl-phenyl]-piperidin-1-yl}-ethanone;

1-(4-{1-acetyl-4-[4-(3,3-dimethyl-2-oxo-butoxy)-3-methyl-phenyl]-piperidin-4-yl}-2-methyl-phenoxy)-3,3-dimethyl-butan-2-one;

3-(4-{1-ethyl-1-[4-(3-hydroxy-3-methylbutyl)-3-methylphenyl]-propyl}-2-methylphenoxy)-propane-1,2(S)-diol;

3-(4-{1-ethyl-1-[4-(3-ethyl-3-hydroxypentyl)-3-methylphenyl]-propyl}-2-methyl-phenoxy)-propane-1,2(S)-diol;

3-(4-{1-ethyl-1-[4-(3-hydroxy-5-methylhexyl)-3-methylphenyl]-propyl}-2-methyl-phenoxy)-propane-1,2(S)-diol;

3-(4-{1-ethyl-1-[4-(3-hydroxy-4-methylpentyl)-3-methylphenyl]-propyl}-2-methyl-phenoxy)-propane-1,2(S)-diol;

3-(2-ethyl-4-{1-ethyl-1-[4-(3-hydroxy-4,4-dimethylpentyl)-3-methylphenyl]-propyl}-phenoxy)-propane-1,2(S)-diol;

3-(4-{1-ethyl-1-[4-(3-hydroxy-4,4-dimethylpentyl)-3-methylphenyl]-propyl}-2-methyl-phenoxy)-propane-1,2(S)-diol;

3-[4-(1-ethyl-1-{4-[3(S)-hydroxy-4,4-dimethylpentyl]-3-methylphenyl}-propyl)-2-methyl-phenoxy]-propane-1,2(S)-diol;

3-[4-(1-ethyl-1-{4-[3(R)-hydroxy-4,4-dimethylpentyl]-3-methylphenyl}-propyl)-2-methyl-phenoxy]-propane-1,2(S)-diol and

3-(4-{1-ethyl-1-[4-(3-hydroxy-4,4-dimethylpentyl)-phenyl]-propyl) -2-methylphenoxy)-propane-1,2(S)-diol.

In another aspect the invention is drawn to methods employing non-secosteroidal vitamin D mimic compounds having Formula II:

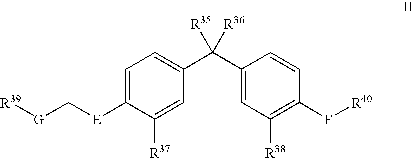

wherein:

E and F are each independently selected from the group consisting of O, S, and NR⁴¹;

G is selected from the group consisting of C═O, CH(OR⁴²), and CH(NR⁴³R⁴⁴);

R³⁵ and R³⁶ are independently selected from the group consisting of alkyl groups, optionally fluorinated; or together R³⁵ and R³⁶ form a cycloalkylidene having 3 to 8 carbon atoms, optionally fluorinated;

R³⁷ and R³⁸ are independently selected from the group consisting of halogen; lower n-alkyl, optionally fluorinated; and lower alkoxy, optionally fluorinated;

R³⁹ is selected from the group consisting of H; optionally substituted alkyl groups; optionally substituted alkenyl groups; optionally substituted alkynyl groups; optionally substituted aryl groups; OR⁴⁵; NR⁴⁶R⁴⁷; or together with R⁴², R⁴³, or R⁴⁴ forms a 3- to 12-membered cyclic group wherein said cyclic group is selected from the group consisting of amidines, amines, ethers, lactams, lactones, ketals, hemiketals, aminals, hemiaminals, carbonates, carbamates, ureas, and combinations thereof;

R⁴⁰ is selected from the group consisting of H and alkyl groups, optionally substituted;

R⁴¹ is selected from the group consisting of H and alkyl groups, optionally substituted;

R⁴² is selected from the group consisting of H, optionally substituted alkyl groups, optionally substituted alkenyl groups, optionally substituted alkynyl groups, optionally substituted aryl group, and optionally substituted acyl groups;

R⁴³ and R⁴⁴ are independently selected from the group consisting of H, optionally substituted alkyl groups, optionally substituted alkenyl groups, optionally substituted alkynyl groups, optionally substituted aryl groups, and optionally substituted acyl groups;

R⁴⁵ is selected from the group consisting of H, optionally substituted alkyl groups, optionally substituted alkenyl groups, optionally substituted alkynyl groups, optionally substituted aryl groups, and optionally substituted acyl groups; and

R⁴⁶ and R⁴⁷ are independently selected from the group consisting of H, optionally substituted alkyl groups, optionally substituted alkenyl groups, optionally substituted alkynyl groups, optionally substituted aryl groups, and optionally substituted acyl groups and pharmaceutically acceptable salts thereof.

In a first embodiment, when K and L are both O, M is C═O, and R⁴⁵ is selected from the group consisting of OH and C₁-C₄ alkoxy, then R⁴⁶ is not carboxymethyl and alkyl esters thereof. In a second embodiment, when K and L are both O, and M is selected from the group consisting of CH(OR⁴⁸) and CH(NR⁴⁹ R⁵⁰), then R⁴⁵ is not H or primary alkyl. In a third embodiment, when K and L are both O, and M is CH(OR⁴⁸), then R⁴⁶ and R⁴⁸ do not both comprise aziridines. In a fourth embodiment, when K and L are both O, and M is CH(OR⁴⁸), then R⁴⁵, R⁴⁶, and R⁴⁸ do not simultaneously comprise alkenyl ethers. In a fifth embodiment, when K and L are both O, and M is CH(OR⁴⁸), then R⁴⁵ and R⁴⁶ do not both comprise glycidyl ethers.

In a preferred embodiment of the invention, the active vitamin D compound has a reduced hypercalcemic effect as compared to vitamin D so that sufficient doses of the compound can be administered without inducing hypercalcemia in the animal. The reduced hypercalcemic effect may be due to the active vitamin D compound itself, the regimen by which the compound is administered, or both. A reduced hypercalcemic effect is defined as an effect which is less than the hypercalcemic effect induced by administration of an equal dose of 1α,25-hydroxyvitamin D₃ (calcitriol). As an example, EB 1089 has a hypercalcemic effect which is 50% of the hypercalcemic effect of calcitriol. Additional active vitamin D compounds having a reduced hypercalcemic effect include Ro23-7553 and Ro24-5531 available from Hoffman LaRoche. Other examples of active vitamin D compounds having a reduced hypercalcemic effect can be found in U.S. Pat. No. 4,717,721. Determining the hypercalcemic effect of an active vitamin D compound is routine in the art and can be carried out as disclosed in Hansen et al., Curr. Pharm. Des. 6:803-828 (2000).

The term “chemotherapeutic agent,” as used herein, is intended to refer to any chemotherapeutic agent known to those of skill in the art to be effective for the treatment or amelioration of cancer. Chemotherapeutic agents include, but are not limited to; small molecules; synthetic drugs; peptides; polypeptides; proteins; nucleic acids (e.g., DNA and RNA nucleotides including, but not limited to, antisense nucleotide sequences, triple helices and nucleotide sequences encoding biologically active proteins, polypeptides or peptides); antibodies; synthetic or natural inorganic molecules; mimetic agents; and synthetic or natural organic molecules. Any agent which is known to be useful, or which has been used or is currently being used for the treatment or amelioration of cancer can be used in combination with an active vitamin D compound in accordance with the invention described herein. See, e.g., Hardman et al., eds., 1996, Goodman & Gilman's The Pharmacological Basis Of Basis Of Therapeutics 9th Ed, Mc-Graw-Hill, New York, N.Y. for information regarding therapeutic agents which have been or are currently being used for the treatment or amelioration of cancer.

Chemotherapeutic agents useful in the methods and compositions of the invention include alkylating agents, antimetabolites, anti-mitotic agents, epipodophyllotoxins, antibiotics, hormones and hormone antagonists, enzymes, platinum coordination complexes, anthracenediones, substituted ureas, methylhydrazine derivatives, imidazotetrazine derivatives, cytoprotective agents, DNA topoisomerase inhibitors, biological response modifiers, retinoids, therapeutic antibodies, differentiating agents, immunomodulatory agents, and angiogenesis inhibitors.

Preferred chemotherapeutic agents include those that have been used, are currently used, or are known to be useful for the treatment or amelioration of lung cancer. Preferred agents include, but are not limited to, cisplatin, carboplatin, paclitaxel, docetaxel, etoposide, vincristine, vinblastine, cyclophosphamide, doxorubicin, vinorelbine, topotecan, gemcitabine, irinotecan, gifitinib, ifosfamide, tarceva, oblimersen, and TLK286.

Other chemotherapeutic agents that may be used include abarelix, aldesleukin, alemtuzumab, alitretinoin, allopurinol, altretamine, amifostine, anastrozole, arsenic trioxide, asparaginase, azacytidine, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, SN-38, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

Chemotherapeutic agents also include anti-inflammatory drugs which are known to be useful for ameliorating inflammation. Suitable anti-inflammatory drugs include, but are not limited to, salicylates (such as aspirin, choline inagnessium trisalicylate, methyl salicylate, salsalte and diflunisal), acetic acids (such as indomethacin, sulindac, tolmetin, aceclofenac and diclofenac), 2-arylpropionic acids or profens (such as ibuprofen, ketoprofen, naproxen, fenoprofen, flurbiprofen and oxaprozin), N-arylanthranilic acids or fenamic acids (such as mefenamic acid, flufenamic acid, and meclofenamate), enolic acids or oxicams (such as piroxicam and meloxicam), cox inhibitors (such as celecoxib, rofecoxib (withdrawn from market), valdecoxib, parecoxib and etoricoxib), sulphonanilides such as nimesulide; naphthylalkanones (such as nabumetone), pyranocarboxylic acids (such as etodolac) and pyrroles (such as ketorolac).

Chemotherapeutic agents further include immunomodulatory agents. As used herein, the term “immunomodulatory agent” and variations thereof including, but not limited to, immunomodulatory agents, immunomodulants, immunomodulators or immunomodulatory drugs, refer to an agent that modulates a host's immune system. In particular, an immunomodulatory agent is an agent that alters the ability of a subject's immune system to respond to one or more foreign antigens. In a specific embodiment, an immunomodulatory agent is an agent that shifts one aspect of a subject's immune response, e.g., the agent shifts the immune response from a Th1 to a Th2 response. In certain embodiments, an immunomodulatory agent is an agent that inhibits or reduces a subject's immune system (i.e., an immunosuppressant agent). In certain other embodiments, an immunomodulatory agent is an agent that activates or increases a subject's immune system (i.e., an immunostimulatory agent).

Immunomodulatory agents useful for the present invention include, but are not limited to, small molecules, peptides, polypeptides, proteins, nucleic acids (e.g., DNA and RNA nucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules. A particularly useful immunomodulatory agent useful for the present invention is thalidomide.

Examples of immunosuppressant agents useful for the present invention include glucocorticoid receptor agonists (e.g., cortisone, dexamethasone, hydrocortisone, betamethasone), calcineurin inhibitors (e.g., macrolides such as tacrolimus and pimecrolimus), immunophilins (e.g., cyclosporin A) and mTOR inhibitors (e.g., sirolimus, marketed as RAPAMUNE® by Wyeth). Immunostimulant agents useful for the present invention include interferon and Zidovudine (AZT).

Chemotherapeutic agents may be administered at doses that are recognized by those of skill in the art to be effective for the treatment of lung cancer. In certain embodiments, chemotherapeutic agents may be administered at doses lower than those used in the art due to the additive or synergistic effect of the active vitamin D compound or a mimic thereof.

The term “radiotherapeutic agent,” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy.

Brachytherapy can be administered according to any schedule, dose, or method known to one of skill in the art to be effective in the treatment or amelioration of cancer, without limitation. In general, brachytherapy comprises insertion of radioactive sources into the body of a subject to be treated for cancer, preferably inside the tumor itself, such that the tumor is maximally exposed to the radioactive source, while preferably minimizing the exposure of healthy tissue. Representative radioisotopes that can be administered in brachytherapy include, but are not limited to, phosphorus 32, cobalt 60, palladium 103, ruthenium 106, iodine 125, cesium 137, iridium 192, xenon 133, radium 226, californium 252, or gold 198. Methods of administering and apparatuses and compositions useful for brachytherapy are described in Mazeron et al., Sem. Rad. Onc. 12:95-108 (2002) and U.S. Pat. Nos. 6,319,189, 6,179,766, 6,168,777, 6,149,889, and 5,611,767, each of which is incorporated herein by reference in its entirety.

Radionuclide therapy can be administered according to any schedule, dose, or method known to one of skill in the art to be effective in the treatment or amelioration of cancer, without limitation. In general, radionuclide therapy comprises systemic administration of a radioisotope that preferentially accumulates in or binds to the surface of cancerous cells. The preferential accumulation of the radionuclide can be mediated by a number of mechanisms, including, but not limited to, incorporation of the radionuclide into rapidly proliferating cells, specific accumulation of the radionuclide by the cancerous tissue without special targeting, or conjugation of the radionuclide to a biomolecule specific for a neoplasm.

Representative radioisotopes that can be administered in radionuclide therapy include, but are not limited to, phosphorus 32, yttrium 90, dysprosium 165, indium 111, strontium 89, samarium 153, rhenium 186, iodine 131, iodine 125, lutetium 177, and bismuth 213. While all of these radioisotopes may be linked to a biomolecule providing specificity of targeting, iodine 131, indium 111, phosphorus 32, samarium 153, and rhenium 186 may be administered systemically without such conjugation. One of skill in the art may select a specific biomolecule for use in targeting a particular neoplasm for radionuclide therapy based upon the cell-surface molecules present on that neoplasm. Examples of biomolecules providing specificity for particular cell are reviewed in an article by Thomas, Cancer Biother. Radiopharm. 17:71-82 (2002), which is incorporated herein by reference in its entirety. Furthermore, methods of administering and compositions useful for radionuclide therapy may be found in U.S. Pat. Nos. 6,426,400, 6,358,194, 5,766,571, and 5,563,250, each of which is incorporated herein by reference in its entirety.

The term “radiotherapeutic treatment,” as used herein, is intended to refer to any radiotherapeutic treatment known to one of skill in the art to be effective to treat or ameliorate cancer, without limitation. For instance, the radiotherapeutic treatment can be external-beam radiation therapy, thermotherapy, radiosurgery, charged-particle radiotherapy, neutron radiotherapy, or photodynamic therapy.

External-beam radiation therapy can be administered according to any schedule, dose, or method known to one of skill in the art to be effective in the treatment or amelioration of cancer, without limitation. In general, external-beam radiation therapy comprises irradiating a defined volume within a subject with a high energy beam, thereby causing cell death within that volume. The irradiated volume preferably contains the entire cancer to be treated, and preferably contains as little healthy tissue as possible. Methods of administering and apparatuses and compositions useful for external-beam radiation therapy can be found in U.S. Pat. Nos. 6,449,336, 6,398,710, 6,393,096, 6,335,961, 6,307,914, 6,256,591, 6,245,005, 6,038,283, 6,001,054, 5,802,136, 5,596,619, and 5,528,652, each of which is incorporated herein by reference in its entirety.

Thermotherapy can be administered according to any schedule, dose, or method known to one of skill in the art to be effective in the treatment or amelioration of cancer, without limitation. In certain embodiments, the thermotherapy can be cryoablation therapy. In other embodiments, the thermotherapy can be hyperthermic therapy. In still other embodiments, the thermotherapy can be a therapy that elevates the temperature of the tumor higher than in hyperthermic therapy.

Cryoablation therapy involves freezing of a neoplastic mass, leading to deposition of intra- and extra-cellular ice crystals; disruption of cellular membranes, proteins, and organelles; and induction of a hyperosmotic environment, thereby causing cell death. Methods for and apparatuses useful in cryoablation therapy are described in Murphy et al., Sem. Urol. Oncol. 19:133-140 (2001) and U.S. Pat. Nos. 6,383,181, 6,383,180, 5,993,444, 5,654,279, 5,437,673, and 5,147,355, each of which is incorporated herein by reference in its entirety.

Hyperthermic therapy typically involves elevating the temperature of a neoplastic mass to a range from about 42° C. to about 44° C. The temperature of the cancer may be further elevated above this range; however, such temperatures can increase injury to surrounding healthy tissue while not causing increased cell death within the tumor to be treated. The tumor may be heated in hyperthermic therapy by any means known to one of skill in the art without limitation. For example, and not by way of limitation, the tumor may be heated by microwaves, high intensity focused ultrasound, ferromagnetic thermoseeds, localized current fields, infrared radiation, wet or dry radiofrequency ablation, laser photocoagulation, laser interstitial thermic therapy, and electrocautery. Microwaves and radiowaves can be generated by waveguide applicators, horn, spiral, current sheet, and compact applicators.

Other methods of and apparatuses and compositions for raising the temperature of a tumor are reviewed in an article by Wust et al., Lancet Oncol. 3:487-97 (2002), and described in U.S. Pat. Nos. 6,470,217, 6,379,347, 6,165,440, 6,163,726, 6,099,554, 6,009,351, 5,776,175, 5,707,401, 5,658,234, 5,620,479, 5,549,639, and 5,523,058, each of which is incorporated herein by reference in its entirety.

Radiosurgery can be administered according to any schedule, dose, or method known to one of skill in the art to be effective in the treatment or amelioration of cancer, without limitation. In general, radiosurgery comprises exposing a defined volume within a subject to a manually directed radioactive source, thereby causing cell death within that volume. The irradiated volume preferably contains the entire cancer to be treated, and preferably contains as little healthy tissue as possible. Typically, the tissue to be treated is first exposed using conventional surgical techniques, then the radioactive source is manually directed to that area by a surgeon. Alternatively, the radioactive source can be placed near the tissue to be irradiated using, for example, a laparoscope. Methods and apparatuses useful for radiosurgery are further described in Valentini et al., Eur. J. Surg. Oncol. 28:180-185 (2002) and in U.S. Pat. Nos. 6,421,416, 6,248,056, and 5,547,454, each of which is incorporated herein by reference in its entirety.

Charged-particle radiotherapy can be administered according to any schedule, dose, or method known to one of skill in the art to be effective in the treatment or amelioration of cancer, without limitation. In certain embodiments, the charged-particle radiotherapy can be proton beam radiotherapy. In other embodiments, the charged-particle radiotherapy can be helium ion radiotherapy. In general, charged-particle radiotherapy comprises irradiating a defined volume within a subject with a charged-particle beam, thereby causing cellular death within that volume. The irradiated volume preferably contains the entire cancer to be treated, and preferably contains as little healthy tissue as possible. A method for administering charged-particle radiotherapy is described in U.S. Pat. No. 5,668,371, which is incorporated herein by reference in its entirety.

Neutron radiotherapy can be administered according to any schedule, dose, or method known to one of skill in the art to be effective in the treatment or amelioration of cancer, without limitation. In certain embodiments, the neutron radiotherapy can be a neutron capture therapy. In such embodiments, a compound that emits radiation when bombarded with neutrons and preferentially accumulates in a neoplastic mass is administered to a subject. Subsequently, the tumor is irradiated with a low energy neutron beam, activating the compound and causing it to emit decay products that kill the cancerous cells. The compound to be activated can be caused to preferentially accumulate in the target tissue according to any of the methods useful for targeting of radionuclides, as described above, or in the methods described in Laramore, Semin. Oncol. 24:672-685 (1997) and in U.S. Pat. Nos. 6,400,796, 5,877,165, 5,872,107, and 5,653,957, each of which is incorporated. herein by reference in its entirety.

In other embodiments, the neutron radiotherapy can be a fast neutron radiotherapy. In general, fast neutron radiotherapy comprises irradiating a defined volume within a subject with a neutron beam, thereby causing cellular death within that volume.

Photodynamic therapy can be administered according to any schedule, dose, or method known to one of skill in the art to be effective in the treatment or amelioration of cancer, without limitation. In general, photodynamic therapy comprises administering a photosensitizing agent that preferentially accumulates in a neoplastic mass and sensitizes the neoplasm to light, then exposing the tumor to light of an appropriate wavelength. Upon such exposure, the photosensitizing agent catalyzes the production of a cytotoxic agent, such as, e.g., singlet oxygen, which kills the cancerous cells. Methods of administering and apparatuses and compositions useful for photodynamic therapy are disclosed in Hopper, Lancet Oncol. 1:212-219 (2000) and U.S. Pat. Nos. 6,283,957, 6,071,908, 6,011,563, 5,855,595, 5,716,595, and 5,707,401, each of which is incorporated herein by reference in its entirety.

While not intending to be bound by any particular theory of operation, it is believed that active vitamin D compounds can enhance the sensitivity of cancerous cells to radiotherapy, and this enhanced sensitivity is due to changes in cell mechanisms regulating apoptosis and/or the cell cycle. Administration of an active vitamin D compound can not only enhance but also expand the applicability of radiotherapy in the treatment or amelioration of cancer, that would otherwise not respond to current radiotherapy. Further, sensitizing cells to treatment can allow use of a lower dose of radiotherapy, which reduces the side effects associated with the radiotherapy.

Radiotherapy can be administered to destroy tumor cells before or after surgery, before or after chemotherapy, and sometimes during chemotherapy. Radiotherapy may also be administered for palliative reasons to relieve symptoms of cancer, for example, to lessen pain. Among the types of tumors that can be treated using radiotherapy are localized tumors that cannot be excised completely and metastases and tumors whose complete excision would cause unacceptable functional or cosmetic defects or be associated with unacceptable surgical risks.

It will be appreciated that both the particular radiation dose to be utilized in treating lung cancer and the method of administration will depend on a variety of factors. Thus, the dosages of radiation that can be used according to the methods of the present invention are determined by the particular requirements of each situation. The dosage will depend on such factors as the size of the tumor, the location of the tumor, the age and sex of the patient, the frequency of the dosage, the presence of other tumors, possible metastases and the like. Those skilled in the art of radiotherapy can readily ascertain the dosage and the method of administration for any particular tumor by reference to Hall, E. J., Radiobiology for the Radiobiologist, 5th edition, Lippincott Williams & Wilkins Publishers, Philadelphia, Pa., 2000; Gunderson, L. L. and Tepper J. E., eds., Clinical Radiation Oncology, Churchill Livingstone, London, England, 2000; and Grosch, D. S., Biological Effects of Radiation, 2nd edition, Academic Press, San Francisco, Calif., 1980, each of which is incorporated herein by reference.

The active vitamin D compound is preferably administered at a dose of about 0.1 μg to about 10 mg, e.g., about 0.5 μg to about 1 mg, or from about 15 μg to about 500 μg. In a specific embodiment, an effective amount of an active vitamin D compound is 0.5, 1, 2, 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 2000, 3000, 4000, 5000, 6000, 7000, 8000, 9000, or 10000 μg or more. In certain embodiments, an effective dose of an active vitamin D compound is between about 3 μg to about 10 mg, e.g., between about 15 μg to about 1 mg, between about 30 μg to about 300 μg, between about 105 μg to about 200 μg, or about 165 μg. In certain embodiments, the methods of the invention comprise administering an active vitamin D compound in a dose of about 0.12 μg/kg bodyweight to about 200 μg/kg bodyweight. The compound may be administered by any route, including oral, intramuscular, intravenous, parenteral, rectal, nasal, topical, or transdermal.

According to the methods of the invention, the active vitamin D compound or a mimic thereof is administered by HDPA so that high doses of the active vitamin D compound can be administered without inducing hypercalcemia. Pulse administration refers to intermittently administering an active vitamin D compound on either a continuous intermittent dosing schedule or a non-continuous intermittent dosing schedule. High doses of active vitamin D compounds include doses greater than about 3 μg as discussed in the sections above. Therefore, the methods for the treatment or amelioration of lung cancer encompass intermittently administering high doses of active vitamin D compounds. The frequency of the HDPA can be limited by a number of factors including, but not limited to, the pharmacokinetic parameters of the compound or formulation and the pharmacodynamic effects of the active vitamin D compound on the animal. For example, animals with lung cancer having impaired renal function may require less frequent administration of the active vitamin D compound because of the decreased ability of those animals to excrete calcium.

The following is exemplary only and merely serves to illustrate that the term HDPA can encompass any discontinuous administration regimen designed by a person of skill in the art.

In one example, the active vitamin D compound or a mimic thereof can be administered not more than once every three days, every four days, every five days, every six days, every seven days, every eight days, every nine days, every ten days, every two weeks, every three weeks, or every four weeks. The administration can continue for one, two, three, or four weeks or one, two, or three months, or longer. Optionally, after a period of rest, the active vitamin D compound can be administered under the same or a different schedule. The period of rest can be one, two, three, or four weeks, or longer, according to the pharmacodynamic effects of the active vitamin D compound on the animal. In another example, the active vitamin D compound can be administered intermittently on a short term daily basis, e.g., once a day for three days, repeated no more frequently than once per week.

In another example, the active vitamin D compound or a mimic thereof can be administered once per week for three months.

In a preferred embodiment, the vitamin D compound or a mimic thereof can be administered once in a three week cycle. After a one week period of rest, the active vitamin D compound can be administered under the same or different schedule.

Further examples of dosing schedules that can be used in the methods of the present invention are provided in U.S. Pat. No. 6,521,608, which is incorporated by reference in its entirety.

The above-described administration schedules are provided for illustrative purposes only and should not be considered limiting. A person of skill in the art will readily understand that all active vitamin D compounds are within the scope of the invention and that the exact dosing and schedule of administration of the active vitamin D compounds can vary due to many factors.

The amount of a therapeutically effective dose of a pharmaceutical agent in the acute or chronic management of a disease or disorder may differ depending on factors including but not limited to the disease or disorder treated, the specific pharmaceutical agents and the route of administration. According to the methods of the invention, an effective dose of an active vitamin D compound or a mimic thereof is any dose of the compound effective to treat or ameliorate lung cancer. A high dose of an active vitamin D compound can be a dose from about 3 μg to about 10 mg or any dose within this range as discussed above. The dose, dose frequency, duration, or any combination thereof, may also vary according to age, body weight, response, and the past medical history of the animal as well as the route of administration, pharmacokinetics, and pharmacodynamic effects of the pharmaceutical agents. These factors are routinely considered by one of skill in the art.

The rates of absorption and clearance of vitamin D compounds are affected by a variety of factors that are well known to persons of skill in the art. As discussed above, the pharmacokinetic properties of active vitamin D compounds limit the peak concentration of vitamin D compounds that can be obtained in the blood without inducing the onset of hypercalcemia. The rate and extent of absorption, distribution, binding or localization in tissues, biotransformation, and excretion of the active vitamin D compound can all affect the frequency at which the pharmaceutical agents can be administered.

In one embodiment of the invention, an active vitamin D compound or a mimic thereof is administered at a dose sufficient to achieve peak plasma concentrations of the active vitamin D compound of about 0.1 nM to about 1000 nM, e.g., about 0.1 nM to about 20 nM. In certain embodiments, the methods of the invention comprise administering the active vitamin D compound in a dose that achieves peak plasma concentrations of 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 12.5 nM, 15 nM, 17.5 nM, 20 nM, 22.5 nM, 25 nM, 30 nM, 35 nM, 40 nM, 45 nM, 50 nM, 60 nM, 70 nM, 80 nM, 90 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM or 1000 nM or any range of concentrations therein. In other embodiments, the active vitamin D compound is administered in a dose that achieves peak plasma concentrations of the active vitamin D compound exceeding about 0.5 nM, e.g., about 0.5 nM to about 1000 nM, about 0.5 nM to about 100 nM, about 0.5 nM to about 25 nM, about 5 nM to about 20 nM, or about 10 nM to about 15 nM.

In another preferred embodiment, the active vitamin D compound or a mimic thereof is administered at a dose of at least about 0.12 μg/kg bodyweight, more preferably at a dose of at least about 0.5 μg/kg bodyweight.

One of skill in the art will recognize that these standard doses are for an average sized adult of approximately 70 kg and can be adjusted for the factors routinely considered as stated above.

In certain embodiments, the methods of the invention further comprise administering a dose of an active vitamin D compound or a mimic thereof that achieves peak plasma concentrations rapidly, e.g., within four hours. In further embodiments, the methods of the invention comprise administering a dose of an active vitamin D compound that is eliminated quickly, e.g., with an elimination half-life of less than 12 hours.

While obtaining high concentrations of the active vitamin D compound is beneficial, it must be balanced with clinical safety, e.g., hypercalcemia. Thus, in one aspect of the invention, the methods of the invention HDPA of active vitamin D compounds to a subject with lung cancer and monitoring the subject for symptoms associated with hypercalcemia. Such symptoms include calcification of soft tissues (e.g., cardiac tissue), increased bone density, and hypercalcemic nephropathy. In still another embodiment, the methods of the invention encompass HDPA of an active vitamin D compound to a subject with lung cancer and monitoring the calcium plasma concentration of the subject to ensure that the calcium plasma concentration is less than about 11.5 mg/dL.

In certain embodiments, high blood levels of vitamin D compounds or mimics thereof can be safely obtained in conjunction with reducing the transport of calcium into the blood. In one embodiment, higher active vitamin D compound concentrations are safely obtainable without the onset of hypercalcemia when administered in conjunction with a reduced calcium diet. In one example, the calcium can be trapped by an adsorbent, absorbent, ligand, chelate, or other binding moiety that cannot be transported into the blood through the small intestine. In another example, the rate of osteoclast activation can be inhibited by administering, for example, a bisphosphonate such as, e.g., zoledronate, pamidronate, or alendronate, or a corticosteroid such as, e.g., dexamethasone or prednisone, in conjunction with the active vitamin D compound.

In certain embodiments, high blood levels of active vitamin D compounds or mimics thereof are safely obtained in conjunction with maximizing the rate of clearance of calcium. In one example, calcium excretion can be increased by ensuring adequate hydration and salt intake. In another example, diuretic therapy can be used to increase calcium excretion.

The doses of the vitamin D analogs and vitamin D mimics may be adjusted proportionate to the ratio of the efficacy index to the calcemic index according to the formula:
Dose=CalcitriolDose×(EI÷CI)
where Dose is the analog or mimic dose, calcitriolDose is calcitriol dose, EI is the analog or mimic efficacy index and CI is the analog or mimic calcemic index, wherein the term “efficacy index” is the ratio of the concentration of the vitamin D analog or mimic to the concentration of calcitriol at equivalent potency. Thus, the efficacy index is a fraction less than one when the vitamin D analog or mimic is less potent than calcitriol. EI is a number greater than one when calcitriol is less potent than the vitamin D analog or mimic. The “calcemic index” of a drug is a measure of the relative ability of the drug to generate a calcemic response as reported in Bouillon et al., Endocrine Rev. 16:200 (1995). A calcemic index of 1 corresponds to the relative calcemic activity of calcitriol. A calcemic index of about 0.01 corresponds to the calcemic activity of a drug with approximately 100 times less calcemic activity than calcitriol. A calcemic index of 0.5 would correspond to a drug having approximately half the calcemic activity of calcitriol. The calcemic index of a drug can vary depending on the assay conducted, e.g., whether one is measuring stimulation of intestinal calcium absorption (a process by which dietary calcium enters into the physiological processes to contribute to the skeletal growth of the organism and to the maintenance of calcium homeostasis) or bone calcium mobilizing activity (a process by which the bone matrix acts as an exchangeable reservoir for calcium). See U.S. Pat. No. 6,521,608 for further detail.

The active vitamin D compound or a mimic thereof may be administered as part of a pharmaceutical composition comprising a pharmaceutically acceptable carrier, wherein the active vitamin D compound is present in an amount which is effective to achieve its intended purpose, i.e., to have an anti-proliferative effect. The pharmaceutical composition may further comprise one or more excipients, diluents or any other components known to persons of skill in the art and germane to the methods of formulation of the present invention. The pharmaceutical composition may additionally comprise other compounds typically used as adjuncts during cancer therapy (e.g., anti-emetics, steroids).

The term “pharmaceutical composition” as used herein is to be understood as defining compositions of which the individual components or ingredients are themselves pharmaceutically acceptable, e.g., where oral administration is foreseen, acceptable for oral use and, where topical administration is foreseen, topically acceptable.

The pharmaceutical composition can be prepared in single unit dosage forms. The dosage forms are suitable for oral, mucosal (nasal, sublingual, vaginal, buccal, rectal), parenteral (intravenous, intramuscular, intraarterial), or topical administration. Preferred dosage forms of the present invention include oral dosage forms and intravenous dosage forms.

Intravenous forms include, but are not limited to, bolus and drip injections. In preferred embodiments, the intravenous dosage forms are sterile or capable of being sterilized prior to administration to a subject since they typically bypass the subject's natural defenses against contaminants. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

In a preferred embodiment of the invention, the pharmaceutical compositions comprising active vitamin D compounds or mimics thereof are emulsion pre-concentrate formulations. The compositions of the invention meet or substantially reduce the difficulties associated with active vitamin D compound therapy hitherto encountered in the art including, in particular, undesirable pharmacokinetic parameters of the compound upon administration to a patient.

According to one aspect of the present invention, a pharmaceutical composition is provided comprising (a) a lipophilic phase component, (b) one or more surfactants, (c) an active vitamin D compound or a mimic thereof; wherein said composition is an emulsion pre-concentrate, which upon dilution with water, in a water to composition ratio of about 1 :1 or more of said water, forms an emulsion having an absorbance of greater than 0.3 at 400 nm. The pharmaceutical composition of the invention may further comprise a hydrophilic phase component.

In another aspect of the invention, a pharmaceutical emulsion composition is provided comprising water (or other aqueous solution) and an emulsion pre-concentrate.

The term “emulsion pre-concentrate,” as used herein, is intended to mean a system capable of providing an emulsion upon contacting with, e.g., water. The term “emulsion,” as used herein, is intended to mean a colloidal dispersion comprising water and organic components including hydrophobic (lipophilic) organic components. The term “emulsion” is intended to encompass both conventional emulsions, as understood by those skilled in the art, as well as “sub-micron droplet emulsions,” as defined immediately below.

The term “sub-micron droplet emulsion,” as used herein is intended to mean a dispersion comprising water and organic components including hydrophobic (lipophilic) organic components, wherein the droplets or particles formed from the organic components have an average maximum dimension of less than about 1000 nm.

Sub-micron droplet emulsions are identifiable as possessing one or more of the following characteristics. They are formed spontaneously or substantially spontaneously when their components are brought into contact, that is without substantial energy supply, e.g., in the absence of heating or the use of high shear equipment or other substantial agitation. They exhibit thermodynamic stability and they are monophasic.

The particles of a sub-micron droplet emulsion may be spherical, though other structures are feasible, e.g., liquid crystals with lamellar, hexagonal or isotropic symmetries. Generally, sub-micron droplet emulsions comprise droplets or particles having a maximum dimension (e.g., average diameter) of between about 50 nm to about 1000 nm, and preferably between about 200 nm to about 300 nm.

The pharmaceutical compositions of the present invention will generally form an emulsion upon dilution with water. The emulsion will form according to the present invention upon the dilution of an emulsion pre-concentrate with water in a water to composition ratio of about 1:1 or more of said water. According to the present invention, the ratio of water to composition can be, e.g., between 1:1 and 5000:1. For example, the ratio of water to composition can be about 1:1, 2:1, 3:1, 4:1, 5:1, 10:1, 200:1, 300:1, 500:1, 1000:1, or 5000:1. The skilled artisan will be able to readily ascertain the particular ratio of water to composition that is appropriate for any given situation or circumstance.

According to the present invention, upon dilution of said emulsion pre-concentrate with water, an emulsion will form having an absorbance of greater than 0.3 at 400 nm. The absorbance at 400 nm of the emulsions formed upon 1:100 dilution of the emulsion pre-concentrates of the present invention can be, e.g., between 0.3 and 4.0. For example, the absorbance at 400 nm can be, e.g., about 0.4, 0.5, 0.6, 1.0, 1.2, 1.6, 2.0, 2.2, 2.4, 2.5, 3.0, or 4.0. Methods for determining the absorbance of a liquid solution are well known by those in the art. The skilled artisan will be able to ascertain and adjust the relative proportions of the ingredients of the emulsions pre-concentrates of the invention in order to obtain, upon dilution with water, an emulsion having any particular absorbance encompassed within the scope of the invention.

The pharmaceutical compositions of the present invention can be, e.g., in a semi-solid formulation or in a liquid formulation. Semi-solid formulations of the present invention can be any semi-solid formulation known by those of ordinary skill in the art, including, e.g., gels, pastes, creams and ointments.

The pharmaceutical compositions of the present invention comprise a lipophilic phase component. Suitable components for use as lipophilic phase components include any pharmaceutically acceptable solvent which is non-miscible with water. Such solvents will appropriately be devoid or substantially devoid of surfactant function.

The lipophilic phase component may comprise mono-, di- or triglycerides. Mono-, di- and triglycerides that may be used within the scope of the invention include those that are derived from C₆, C₈, C₁₀, C₁₂, C₁₄, C₁₆, C₁₈, C₂₀ and C₂₂ fatty acids. Exemplary diglycerides include, in particular, diolein, dipalmitolein, and mixed caprylin-caprin diglycerides. Preferred triglycerides include vegetable oils, fish oils, animal fats, hydrogenated vegetable oils, partially hydrogenated vegetable oils, synthetic triglycerides, modified triglycerides, fractionated triglycerides, medium and long-chain triglycerides, structured triglycerides, and mixtures thereof.

Among the above-listed triglycerides, preferred triglycerides include: almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially soy and cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate.

A preferred triglyceride is the medium chain triglyceride available under the trade name LABRAFAC CC. Other preferred triglycerides include neutral oils, e.g., neutral plant oils, in particular fractionated coconut oils such as known and commercially available under the trade name MIGLYOL, including the products: MIGLYOL 810; MIGLYOL 812; MIGLYOL 818; and CAPTEX 355.

Also suitable are caprylic-capric acid triglycerides such as known and commercially available under the trade name MYRITOL, including the product MYRITOL 813. Further suitable products of this class are CAPMUL MCT, CAPTEX 200, CAPTEX 300, CAPTEX 800, NEOBEE M5 and MAZOL 1400.

Especially preferred as lipophilic phase component is the product MIGLYOL 812. (See U.S. Pat. No. 5,342,625).

Pharmaceutical compositions of the present invention may further comprise a hydrophilic phase component. The hydrophilic phase component may comprise, e.g., a pharmaceutically acceptable C₁₋₅ alkyl or tetrahydrofurfuryl di- or partial-ether of a low molecular weight mono- or poly-oxy-alkanediol. Suitable hydrophilic phase components include, e.g., di- or partial-, especially partial-, -ethers of mono- or poly-, especially mono- or di-, -oxy-alkanediols comprising from 2 to 12, especially 4 carbon atoms. Preferably the mono- or poly-oxy-alkanediol moiety is straight-chained. Exemplary hydrophilic phase components for use in relation to the present invention are those known and commercially available under the trade names TRANSCUTOL and COLYCOFUROL. (See U.S. Pat. No. 5,342,625).

In an especially preferred embodiment, the hydrophilic phase component comprises 1,2-propyleneglycol.

The hydrophilic phase component of the present invention may of course additionally include one or more additional ingredients. Preferably, however, any additional ingredients will comprise materials in which the active vitamin D compound or a mimic thereof is sufficiently soluble, such that the efficacy of the hydrophilic phase as an active vitamin D compound carrier medium is not materially impaired. Examples of possible additional hydrophilic phase components include lower (e.g., C₁₋₅) alkanols, in particular ethanol.

Pharmaceutical compositions of the present invention also comprise one or more surfactants. Surfactants that can be used in conjunction with the present invention include hydrophilic or lipophilic surfactants, or mixtures thereof. Especially preferred are non-ionic hydrophilic and non-ionic lipophilic surfactants.

Suitable hydrophilic surfactants include reaction products of natural or hydrogenated vegetable oils and ethylene glycol, i.e. polyoxyethylene glycolated natural or hydrogenated vegetable oils, for example polyoxyethylene glycolated natural or hydrogenated castor oils. Such products may be obtained in known manner, e.g., by reaction of a natural or hydrogenated castor oil or fractions thereof with ethylene oxide, e.g., in a molar ratio of from about 1:35 to about 1:60, with optional removal of free polyethyleneglycol components from the product, e.g., in accordance with the methods disclosed in German Auslegeschriften 1,182,388 and 1,518,819.

Suitable hydrophilic surfactants for use in the present pharmaceutical compounds also include polyoxyethylene-sorbitan-fatty acid esters, e.g., mono- and trilauryl, palmityl, stearyl and oleyl esters, e.g., of the type known and commercially available under the trade name TWEEN; including the products:

TWEEN 20 (polyoxyethylene(20)sorbitanmonolaurate),

TWEEN 40 (polyoxyethylene(20)sorbitanmonopalmitate),

TWEEN 60 (polyoxyethylene(20)sorbitanmonostearate),

TWEEN 80 (polyoxyethylene(20)sorbitanmonooleate),

TWEEN 65 (polyoxyethylene(20)sorbitantristearate),

TWEEN 85 (polyoxyethylene(20)sorbitantrioleate),

TWEEN 21 (polyoxyethylene(4)sorbitanmonolaurate),

TWEEN 61 (polyoxyethylene(4)sorbitanmonostearate), and

TWEEN 81 (polyoxyethylene(5)sorbitamnonooleate).

Especially preferred products of this class for use in the compositions of the invention are the above products TWEEN 40 and TWEEN 80. (See Hauer, et al., U.S. Pat. No.5,342,625).

Also suitable as hydrophilic surfactants for use in the present pharmaceutical compounds are polyoxyethylene alkylethers; polyoxyethylene glycol fatty acid esters, for example polyoxythylene stearic acid esters; polyglycerol fatty acid esters; polyoxyethylene glycerides; polyoxyethylene vegetable oils; polyoxyethylene hydrogenated vegetable oils; reaction mixtures of polyols and, e.g., fatty acids, glycerides, vegetable oils, hydrogenated vegetable oils, and sterols; polyoxyethylene-polyoxypropylene co-polymers; polyoxyethylene-polyoxypropylene block co-polymers; dioctylsuccinate, dioctylsodiumsulfosuccinate, di-[2-ethylhexyl]-succinate or sodium lauryl sulfate; phospholipids, in particular lecithins such as, e.g., soya bean lecithins; propylene glycol mono- and di-fatty acid esters such as, e.g., propylene glycol dicaprylate, propylene glycol dilaurate, propylene glycol hydroxystearate, propylene glycol isostearate, propylene glycol laurate, propylene glycol ricinoleate, propylene glycol stearate, and, especially preferred, propylene glycol caprylic-capric acid diester; and bile salts, e.g., alkali metal salts, for example sodium taurocholate.

Suitable lipophilic surfactants include alcohols; polyoxyethylene alkylethers; fatty acids; bile acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid esters of mono/diglycerides; propylene glycol diglycerides; sorbitan fatty acid esters; polyoxyethylene sorbitan fatty acid esters; polyoxyethylene-polyoxypropylene block copolymers; trans-esterified vegetable oils; sterols; sugar esters; sugar ethers; sucroglycerides; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; and mixtures thereof.

Suitable lipophilic surfactants for use in the present pharmaceutical compounds also include trans-esterification products of natural vegetable oil triglycerides and polyalkylene polyols. Such trans-esterification products are known in the art and may be obtained e.g., in accordance with the general procedures described in U.S. Pat. No. 3,288,824. They include trans-esterification products of various natural (e.g., non-hydrogenated) vegetable oils for example, maize oil, kernel oil, almond oil, ground nut oil, olive oil and palm oil and mixtures thereof with polyethylene glycols, in particular polyethylene glycols having an average molecular weight of from 200 to 800. Preferred are products obtained by trans-esterification of 2 molar parts of a natural vegetable oil triglyceride with one molar part of polyethylene glycol (e.g., having an average molecular weight of from 200 to 800). Various forms of trans-esterification products of the defined class are known and commercially available under the trade name LABRAFIL.

Additional lipophilic surfactants that are suitable for use with the present pharmaceutical compositions include oil-soluble vitamin derivatives, e.g., tocopherol PEG-1000 succinate (“vitamin E TPGS”).

Also suitable as lipophilic surfactants for use in the present pharmaceutical compounds are mono-, di- and mono/di-glycerides, especially esterification products of caprylic or capric acid with glycerol; sorbitan fatty acid esters; pentaerythritol fatty acid esters and polyalkylene glycol ethers, for example pentaerythrite- -dioleate, -distearate, -monolaurate, -polyglycol ether and -monostearate as well as pentaerythrite-fatty acid esters; monoglycerides, e.g., glycerol monooleate, glycerol monopalmitate and glycerol monostearate; glycerol triacetate or (1,2,3)-triacetin; and sterols and derivatives thereof, for example cholesterols and derivatives thereof, in particular phytosterols, e.g., products comprising sitosterol, campesterol or stigmasterol, and ethylene oxide adducts thereof, for example soya sterols and derivatives thereof.

It is understood by those of ordinary skill in the art that several commercial surfactant compositions contain small to moderate amounts of triglycerides, typically as a result of incomplete reaction of a triglyceride starting material in, for example, a trans-esterification reaction. Thus, the surfactants that are suitable for use in the present pharmaceutical compositions include those surfactants that contain a triglyceride. Examples of commercial surfactant compositions containing triglycerides include some members of the surfactant families GELUCRIES, MAISINES, and IMWITORS. Specific examples of these compounds are GELUCIRE 44/14 (saturated polyglycolized glycerides); GELUCIRE 50/13 (saturated polyglycolized glycerides); GELUCIRE 53/10 (saturated polyglycolized glycerides); GELUCIRE 33/01 (semi-synthetic triglycerides of C₈-C₁₈ saturated fatty acids); GELUCIRE 39/01 (semi-synthetic glycerides); other GELUCIRES, such as 37/06, 43/01, 35/10, 37/02, 46/07, 48/09, 50/02, 62/05, etc.; MAISNE 35-I (linoleic glycerides); and IMWITOR 742 (caprylic/capric glycerides). (See U.S. Pat. No. 6,267,985).

Still other commercial surfactant compositions having significant triglyceride content are known to those skilled in the art. It should be appreciated that such compositions, which contain triglycerides as well as surfactants, may be suitable to provide all or part of the lipophilic phase component of the of the present invention, as well as all or part of the surfactants.

The relative proportion of ingredients in the compositions of the invention will, of course, vary considerably depending on the particular type of composition concerned. The relative proportions will also vary depending on the particular function of ingredients in the composition. The relative proportions will also vary depending on the particular ingredients employed and the desired physical characteristics of the product composition, e.g., in the case of a composition for topical use, whether this is to be a free flowing liquid or a paste. Determination of workable proportions in any particular instance will generally be within the capability of a person of ordinary skill in the art. All indicated proportions and relative weight ranges described below are accordingly to be understood as being indicative of preferred or individually inventive teachings only and not as limiting the invention in its broadest aspect.

The lipophilic phase component of the invention will suitably be present in an amount of from about 30% to about 90% by weight based upon the total weight of the composition. Preferably, the lipophilic phase component is present in an amount of from about 50% to about 85% by weight based upon the total weight of the composition.

The surfactant or surfactants of the invention will suitably be present in an amount of from about 1% to 50% by weight based upon the total weight of the composition. Preferably, the surfactant(s) is present in an amount of from about 5% to about 40% by weight based upon the total weight of the composition.

The amount of active vitamin D compound or a mimic thereof in compositions of the invention will of course vary, e.g., depending on the intended route of administration and to what extent other components are present. In general, however, the active vitamin D compound of the invention will suitably be present in an amount of from about 0.005% to 20% by weight based upon the total weight of the composition. Preferably, the active vitamin D compound is present in an amount of from about 0.01% to 15% by weight based upon the total weight of the composition.

The hydrophilic phase component of the invention will suitably be present in an amount of from about 2% to about 20% by weight based upon the total weight of the composition. Preferably, the hydrophilic phase component is present in an amount of from about 5% to 15% by weight based upon the total weight of the composition.

The pharmaceutical composition of the invention may be in a semisolid formulation. Semisolid formulations within the scope of the invention may comprise, e.g., a lipophilic phase component present in an amount of from about 60% to about 80% by weight based upon the total weight of the composition, a surfactant present in an amount of from about 5% to about 35% by weight based upon the total weight of the composition, and an active vitamin D compound present in an amount of from about 0.01% to about 15% by weight based upon the total weight of the composition.

The pharmaceutical compositions of the invention may be in a liquid formulation. Liquid formulations within the scope of the invention may comprise, e.g., a lipophilic phase component present in an amount of from about 50% to about 60% by weight based upon the total weight of the composition, a surfactant present in an amount of from about 4% to about 25% by weight based upon the total weight of the composition, an active vitamin D compound present in an amount of from about 0.01% to about 15% by weight based upon the total weight of the composition, and a hydrophilic phase component present in an amount of from about 5% to about 10% by weight based upon the total weight of the composition.

Additional compositions that may be used include the following, wherein the percentage of each component is by weight based upon the total weight of the composition excluding the active vitamin D compound or a mimic thereof:

| | | |
|---|---|---|
| a. | Gelucire 44/14 | about 50% |
| | Miglyol 812 | about 50%; |
| b. | Gelucire 44/14 | about 50% |
| | Vitamin E TPGS | about 10% |
| | Miglyol 812 | about 40%; |
| c. | Gelucire 44/14 | about 50% |
| | Vitamin E TPGS | about 20% |
| | Miglyol 812 | about 30%; |
| d. | Gelucire 44/14 | about 40% |
| | Vitamin E TPGS | about 30% |
| | Miglyol 812 | about 30%; |
| e. | Gelucire 44/14 | about 40% |
| | Vitamin E TPGS | about 20% |
| | Miglyol 812 | about 40%; |
| f. | Gelucire 44/14 | about 30% |
| | Vitamin E TPGS | about 30% |
| | Miglyol 812 | about 40%; |
| g. | Gelucire 44/14 | about 20% |
| | Vitamin E TPGS | about 30% |
| | Miglyol 812 | about 50%; |
| h. | Vitamin E TPGS | about 50% |
| | Miglyol 812 | about 50%; |
| i. | Gelucire 44/14 | about 60% |
| | Vitamin E TPGS | about 25% |
| | Miglyol 812 | about 15%; |
| j. | Gelucire 50/13 | about 30% |
| | Vitamin E TPGS | about 5% |
| | Miglyol 812 | about 65%; |
| k. | Gelucire 50/13 | about 50% |
| | Miglyol 812 | about 50%; |
| l. | Gelucire 50/13 | about 50% |
| | Vitamin E TPGS | about 10% |
| | Miglyol 812 | about 40%; |
| m. | Gelucire 50/13 | about 50% |
| | Vitamin E TPGS | about 20% |
| | Miglyol 812 | about 30%; |
| n. | Gelucire 50/13 | about 40% |
| | Vitamin E TPGS | about 30% |
| | Miglyol 812 | about 30%; |
| o. | Gelucire 50/13 | about 40% |
| | Vitamin E TPGS | about 20% |
| | Miglyol 812 | about 40%; |
| p. | Gelucire 50/13 | about 30% |
| | Vitamin E TPGS | about 30% |
| | Miglyol 812 | about 40%; |
| q. | Gelucire 50/13 | about 20% |
| | Vitamin E TPGS | about 30% |
| | Miglyol 812 | about 50%; |
| r. | Gelucire 50/13 | about 60% |
| | Vitamin E TPGS | about 25% |
| | Miglyol 812 | about 15%; |
| s. | Gelucire 44/14 | about 50% |
| | PEG 4000 | about 50%; |
| t. | Gelucire 50/13 | about 50% |
| | PEG 4000 | about 50%; |
| u. | Vitamin E TPGS | about 50% |
| | PEG 4000 | about 40%; |
| v. | Gelucire 44/14 | about 33.3% |
| | Vitamin E TPGS | about 33.3% |
| | PEG 4000 | about 33.3%; |
| w. | Gelucire 50/13 | about 33.3% |
| | Vitamin E TPGS | about 33.3% |
| | PEG 4000 | about 33.3%; |
| x. | Gelucire 44/14 | about 50% |
| | Vitamin E TPGS | about 50%; |
| y. | Gelucire 50/13 | about 50% |
| | Vitamin E TPGS | about 50%; |
| z. | Vitamin E TPGS | about 5% |
| | Miglyol 812 | about 95%; |
| aa. | Vitamin E TPGS | about 5% |
| | Miglyol 812 | about 65% |
| | PEG 4000 | about 30%; |
| ab. | Vitamin E TPGS | about 10% |
| | Miglyol 812 | about 90%; |
| ac. | Vitamin E TPGS | about 5% |
| | Miglyol 812 | about 85% |
| | PEG 4000 | about 10%; and |
| ad. | Vitamin E TPGS | about 10% |
| | Miglyol 812 | about 80% |
| | PEG 4000 | about 10%. |

In one embodiment of the invention, the pharmaceutical compositions comprise an active vitamin D compound or a mimic thereof, a lipophilic component, and a surfactant. The lipophilic component may be present in any percentage from about 1% to about 100%. The lipophilic component may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. The surfactant may be present in any percentage from about 1% to about 100%. The surfactant may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In one embodiment, the lipophilic component is MIGLYOL 812 and the surfactant is vitamin E TPGS. In preferred embodiments, the pharmaceutical compositions comprise about 50% MIGLYOL 812 and about 50% vitamin E TPGS, about 90% MIGLYOL 812 and about 10% vitamin E TPGS, or about 95% MIGLYOL 812 and about 5% vitamin E TPGS.

In another embodiment of the invention, the pharmaceutical compositions comprise an active vitamin D compound or a mimic thereof and a lipophilic component, e.g., around 100% MIGLYOL 812.

In a preferred embodiment, the pharmaceutical compositions comprise about 50% MIGLYOL 812, about 50% vitamin E TPGS, and small amounts of BHA and BHT. This formulation has been shown to be unexpectedly stable, both chemically and physically (see Example 3). The enhanced stability provides the compositions with a longer shelf life. Importantly, the stability also allows the compositions to be stored at room temperature, thereby avoiding the complication and cost of storage under refrigeration. Additionally, this composition is suitable for oral administration and has been shown to be capable of solubilizing high doses of active vitamin D compound, thereby enabling high dose pulse administration of active vitamin D compounds for the treatment of hyperproliferative diseases and other disorders.

In certain embodiments, the pharmaceutical compositions comprise about 50% MIGLYOL 812, about 50% vitamin E TPGS, and about 0.01% to about 0.50% each of BHA and BHT. In other embodiments, the pharmaceutical compositions comprise about 50% MIGLYOL 812, about 50% vitamin E TPGS, and about 0.05% to about 0.35% each of BHA and BHT. In certain embodiments, the pharmaceutical compositions comprise about 50% MIGLYOL 812, about 50% vitamin E TPGS, about 0.35% BHA, and about 0.10% BHT.

Additional compositions that may be used include the following, wherein the percentage of each component is by weight based upon the total weight of the composition excluding the active vitamin D compound or a mimic thereof:

| | | |
|---|---|---|
| a. | Miglyol 812 | about 100% |
| | BHA | about 0.05% |
| | BHT | about 0.05%; |
| b. | Miglyol 812 | about 100% |
| | BHA | about 0.35% |
| | BHT | about 0.10%; |
| c. | Miglyol 812 | about 50% |
| | Vitamin E TPGS | about 50% |
| | BHA | about 0.05% |
| | BHT | about 0.05%; |
| d. | Miglyol 812 | about 50% |
| | Vitamin E TPGS | about 50% |
| | BHT | about 0.10%; |
| e. | Miglyol 812 | about 50% |
| | Vitamin E TPGS | about 50% |
| | BHA | about 0.35%; |
| f. | Miglyol 812 | about 50% |
| | Vitamin E TPGS | about 50% |
| | BHA | about 0.35% |
| | BHT | about 0.10%; and |
| g. | Miglyol 812 | about 50% |
| | Vitamin E TPGS | about 50% |
| | BHA | about 0.28% |
| | BHT | about 0.08%. |

It will be understood by those of skill in the art that the formulations of the invention comprising a lipophilic component and a surfactant in amounts that total about 100% (e.g., about 50% lipophilic component and about 50% surfactant) provide adequate room for the active vitamin D compound and additives (e.g., antioxidants) which are present in the formulation in small amounts, each generally present at less than 1% by weight.

The pharmaceutical compositions comprising the active vitamin D compound or a mimic thereof of the present invention may further comprise one or more additives. Additives that are well known in the art include, e.g., detackifiers, anti-foaming agents, buffering agents, antioxidants (e.g., ascorbyl palmitate, butyl hydroxy anisole (BHA), butyl hydroxy toluene (BHT) and tocopherols, e.g., a-tocopherol (vitamin E)), preservatives, chelating agents, viscomodulators, tonicifiers, flavorants, colorants odorants, opacifiers, suspending agents, binders, fillers, plasticizers, lubricants, and mixtures thereof. The amounts of such additives can be readily determined by one skilled in the art, according to the particular properties desired. For example, antioxidants such as BHA and BHT may each be present in an amount of from about 0.01% to about 0.50% by weight based upon the total weight of the composition, e.g., about 0.05 to about 0.35% by weight, e.g., about 0.01, 0.05, 0.10, 0. 15, 0.20, 0.25, 0.30, 0.35, 0.40, 0.45, or 0.50% by weight.

The additive may also comprise a thickening agent. Suitable thickening agents may be of those known and employed in the art, including, e.g., pharmaceutically acceptable polymeric materials and inorganic thickening agents. Exemplary thickening agents for use in the present pharmaceutical compositions include polyacrylate and polyacrylate co-polymer resins, for example poly-acrylic acid and poly-acrylic acid/methacrylic acid resins; celluloses and cellulose derivatives including: alkyl celluloses, e.g., methyl-, ethyl- and propyl-celluloses; hydroxyalkyl-celluloses, e.g., hydroxypropyl-celluloses and hydroxypropylalkyl-celluloses such as hydroxypropyl-methyl-celluloses; acylated celluloses, e.g., cellulose-acetates, cellulose-acetatephthallates, cellulose-acetatesuccinates and hydroxypropylmethyl-cellulose phthallates; and salts thereof such as sodium-carboxymethyl-celluloses; polyvinylpyrrolidones, including for example poly-N-vinylpyrrolidones and vinylpyrrolidone co-polymers such as vinylpyrrolidone-vinylacetate co-polymers; polyvinyl resins, e.g., including polyvinylacetates and alcohols, as well as other polymeric materials including gum traganth, gum arabicum, alginates, e.g., alginic acid, and salts thereof, e.g., sodium alginates; and inorganic thickening agents such as atapulgite, bentonite and silicates including hydrophilic silicon dioxide products, e.g., alkylated (for example methylated) silica gels, in particular colloidal silicon dioxide products.

Such thickening agents as described above may be included, e.g. to provide a sustained release effect. However, where oral administration is intended, the use of thickening agents as aforesaid will generally not be required and is generally less preferred. Use of thickening agents is, on the other hand, indicated, e.g., where topical application is foreseen.

Compositions in accordance with the present invention may be employed for administration in any appropriate manner, e.g., orally, e.g., in unit dosage form, for example in a solution, in hard or soft encapsulated form including gelatin encapsulated form, parenterally or topically, e.g., for application to the skin, for example in the form of a cream, paste, lotion, gel, ointment, poultice, cataplasm, plaster, dermal patch or the like, or for ophthalmic application, for example in the form of an eye-drop, -lotion or -gel formulation. Readily flowable forms, for example solutions and emulsions, may also be employed e.g., for intralesional injection, or may be administered rectally, e.g., as an enema.

When the composition of the present invention is formulated in unit dosage form, the active vitamin D compound or a mimic thereof will preferably be present in an amount of between 1 and 1000 μg per unit dose. More preferably, the amount of active vitamin D compound per unit dose will be about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 250, 300, 350, 400, 450, 500, 550, 600, 650, 700, 750, 800, 850, 900, 950, or 1000 μg or any amount therein. In one embodiment, the amount of active vitamin D compound per unit dose will be about 5 μg to about 180 μg, more preferably about 10 μg to about 135 μg, more preferably about 45 μg. In one embodiment, the unit dosage form comprises 45, 90, 135, or 180 μg of calcitriol.

When the unit dosage form of the composition is a capsule, the total quantity of ingredients present in the capsule is preferably about 10-1000 μL. More preferably, the total quantity of ingredients present in the capsule is about 100-300 μL. In another embodiment, the total quantity of ingredients present in the capsule is preferably about 10-1500 mg, preferably about 100-1000 mg. In one embodiment, the total quantity is about 225, 450, 675, or 900 mg. In one embodiment, the unit dosage form is a capsule comprising 45, 90, 135, or 180 μg of calcitriol.

The dosage amounts and frequencies of administration of the additional therapeutic agents provided herein are encompassed by the terms therapeutically effective. The dosage and frequency of these agents further will typically vary according to factors specific for each patient depending on the specific therapeutic agents administered, the severity and type of lung cancer, the route of administration, as well as age, body weight, response and the past medical history of the patient. Suitable regimens can be selected by one skilled in the art by considering such factors and by following, for example, dosages reported in the literature and recommended in the Physician's Desk Reference (56^{th} ed., 2002).

For animals that have resectable lung cancer, the active vitamin D compound or a mimic thereof can be administered prior to and/or after surgery. Similarly, the chemotherapeutic agents and radiotherapeutic agents or treatments can be administered prior to and/or after surgery.

Any period of treatment with the active vitamin D compound or a mimic thereof prior to, during or after the administration of the chemotherapeutic agents or radiotherapeutic agents or treatments can be employed in the present invention. The exact period for treatment with the active vitamin D compound will vary depending upon the active vitamin D compound used, the type of lung cancer, the patient, and other related factors. The active vitamin D compound may be administered as little as 12 hours and as much as 3 months prior to or after the administration of the chemotherapeutic agents or radiotherapeutic agents or treatments. The active vitamin D may be administered at least one day before or after administration of the chemotherapeutic agents or radiotherapeutic agents or treatments and for as long as 3 months before or after administration of the chemotherapeutic agents or radiotherapeutic agents or treatments. In certain embodiments, the methods of the invention comprise administering the active vitamin D compound once every 3, 4, 5, 6, 7, 8, 9, or 10 days for a period of 3 days to 60 days before or after administration of the chemotherapeutic agents or radiotherapeutic agents or treatments.

The administration of the active vitamin D compound or a mimic thereof may be continued concurrently with the administration of the chemotherapeutic agents or radiotherapeutic agents or treatments. Additionally, the administration of the active vitamin D compound may be continued beyond the administration of the chemotherapeutic agents or radiotherapeutic agents or treatments.

In certain embodiments of the invention, the method of administering an active vitamin D compound or a mimic thereof alone or in combination with chemotherapeutic agents or radiotherapeutic agents or treatments may be repeated at least once. The method my be repeated as many times as necessary to achieve or maintain a therapeutic response, e.g., from one to about ten times. With each repetition of the method the active vitamin D compound and the chemotherapeutic agents or radiotherapeutic agents or treatments may be the same or different from that used in the previous repetition. Additionally, the time period of administration of the active vitamin D compound and the manner in which it is administered can vary from repetition to repetition.

Animals which may be treated according to the present invention include all animals which may benefit from administration of the compounds of the present invention. Such animals include humans, pets such as dogs and cats, and veterinary animals such as cows, pigs, sheep, goats and the like.

### EXAMPLE 1

### Preparation of Semi-Solid Calcitriol Formulations

Five semi-solid calcitriol formulations (SS1-SS5) were prepared containing the ingredients listed in Table 1. The final formulation contains 0.208 mg calcitriol per gram of semi-solid formulation.

TABLE 1
Composition of Semi-Solid Calcitriol Formulation

| Ingredients | SS1 | SS2 | SS3 | SS4 | SS5 |
|---|---|---|---|---|---|
| Calcitriol | 0.0208 | 0.0208 | 0.0208 | 0.0208 | 0.0208 |
| Miglyol 812 | 80.0 | 0 | 65.0 | 0 | 79.0 |
| Captex 200 | 0 | 82.0 | 0 | 60.0 | 0 |
| Labrafac CC | 0 | 0 | 0 | 0 | 12.0 |
| Vitamin-E TPGS | 20.0 | 18.0 | 5.0 | 5.0 | 9.0 |
| Labrifil M | 0 | 0 | 0 | 0 | 0 |
| Gelucire 44/14 | 0 | 0 | 30.0 | 35.0 | 0 |
| BHT | 0.05 | 0.05 | 0.05 | 0.05 | 0.05 |
| BHA | 0.05 | 0.05 | 0.05 | 0.05 | 0.05 |

Amounts shown are in grams.

1. Preparation of Vehicles

One hundred gram quantities of the five semi-solid calcitriol formulations (SS1-SS5) listed in Table 1 were prepared as follows.

The listed ingredients, except for calcitriol, were combined in a suitable glass container and mixed until homogenous. Vitamin E TPGS and GELUCIRE 44/14 were heated and homogenized at 60° C. prior to weighing and adding into the formulation.

2. Preparation of Active Formulations

The semi-solid vehicles were heated and homogenized at ≦60° C. Under subdued light, 12±1 mg of calcitriol was weighed out into separate glass bottles with screw caps, one bottle for each formulation. (Calcitriol is light sensitive; subdued light/red light should be used when working with calcitriol/calcitriol formulations.) The exact weight was recorded to 0.1 mg. The caps were then placed on the bottles as soon as the calcitriol had been placed into the bottles. Next, the amount of each vehicle required to bring the concentration to 0.208 mg/g was calculated using the following formula:
C_{w}/0.208=required weight of vehicle

Where C_{w}=weight of calcitriol, in mg, and

0.1208=final concentration of calcitriol (mg/g).

Finally, the appropriate amount of each vehicle was added to the respective bottle containing the calcitriol. The formulations were heated (≦60° C.) while being mixed to dissolve the calcitriol.

### EXAMPLE 2

### Dose Ranging Study of Calcitriol and Docetaxel in Patients with Advanced Lung Cancer

Patients having advanced NSCLC (Stage EB or IV) that has progressed on or after platinum-based therapy will be treated with a combination of calcitriol and docetaxel. The drugs will be administered in repeated three week cycles. On day one of each cycle patients will take calcitriol orally at a dose of 45 μg, 75 μg, or 105 μg in the semi-solid #3 formulation described above. On day 2 of each cycle patients will be administered docetaxel at a dose of 75 mg/m² intravenously over one hour. Patients will be premedicated with oral dexamethasone 8 mg BID for 3 days starting one day prior to docetaxel administration in order to reduce the incidence and severity of fluid retention as well as the severity of hypersensitivity reactions. Cycles will be continued for two years depending on the survival duration of the patients. Patients will be monitored for safety by noting adverse events. Patients will be monitored for efficacy be measuring tumor response, progression of disease, duration of response and overall survival.

### EXAMPLE 3

### Stable Unit Dose Formulations

Formulations of calcitriol were prepared to yield the compositions in Table 2. The Vitamin E TPGS was warmed to approximately 50° C. and mixed in the appropriate ratio with MIGLYOL 812. BHA and BHT were added to each formulation to achieve 0.35% w/w of each in the final preparations.

TABLE 2
Calcitriol formulations

| | MIGLYOL | Vitamin E TPGS |
|---|---|---|
| Formulation # | (% wt/wt) | (% wt/wt) |
| 1 | 100 | 0 |
| 2 | 95 | 5 |
| 3 | 90 | 10 |
| 4 | 50 | 50 |

After formulation preparation, Formulations 2-4 were heated to approximately 50° C. and mixed with calcitriol to produce 0.1 μg calcitriol/mg total formulation. The formulations contained calcitriol were then added (˜250 μL) to a 25 mL volumetric flask and deionized water was added to the 25 mL mark. The solutions were then vortexed and the absorbance of each formulation was measured at 400 nm immediately after mixing (initial) and up to 10 min after mixing. As shown in Table 3, all three formulations produced an opalescent solution upon mixing with water. Formulation 4 appeared to form a stable suspension with no observable change in absorbance at 400 nm after 10 min.

TABLE 3
Absorption of formulations suspended in water

Absorbance
at 400 nm

| Formulation # | Initial | 10 min |
|---|---|---|
| 2 | 0.7705 | 0.6010 |
| 3 | 1.2312 | 1.1560 |
| 4 | 3.1265 | 3.1265 |

To further assess the formulations of calcitriol, a solubility study was conducted to evaluate the amount of calcitriol soluble in each formulation. Calcitriol concentrations from 0.1 to 0.6 μg calcitriol/mg formulation were prepared by heating the formulations to 50° C. followed by addition of the appropriate mass of calcitriol. The formulations were then allowed to cool to room temperature and the presence of undissolved calcitriol was determined by a light microscope with and without polarizing light. For each formulation, calcitriol was soluble at the highest concentration tested, 0.6 μg calcitriol/mg formulation.

A 45 μg calcitriol dose is currently being used in Phase 2 human clinical trials. To develop a capsule with this dosage each formulation was prepared with 0.2 μg calcitriol/mg formulation and 0.35% w/w of both BHA and BHT. The bulk formulation mixtures were filled into Size 3 hard gelatin capsules at a mass of 225 mg (45 μg calcitriol). The capsules were then analyzed for stability at 5° C., 25° C./60% relative humidity (RH), 30° C./65% RH, and 40° C./75% RH. At the appropriate time points, the stability samples were analyzed for content of intact calcitriol and dissolution of the capsules. The calcitriol content of the capsules was determined by dissolving three opened capsules in 5 mL of methanol and held at 5° C. prior to analysis. The dissolved samples were then analyzed by reversed phase HPLC. A Phemonex Hypersil BDS C18 column at 30° C. was used with a gradient of acetonitrile from 55% acetonitrile in water to 95% acetonitrile at a flow rate of 1.0 mL/min during elution. Peaks were detected at 265 nm and a 25 μL sample was injected for each run. The peak area of the sample was compared to a reference standard to calculate the calcitriol content as reported in Table 4. The dissolution test was performed by placing one capsule in each of six low volume dissolution containers with 50 mL of deionized water containing 0.5% sodium dodecyl sulfate. Samples were taken at 30, 60 and 90 min after mixing at 75 rpm and 37 ° C. Calcitriol content of the samples was determined by injection of 100 μL samples onto a Betasil C18 column operated at 1 mL/min with a mobile phase of 50:40:10 acetonitrile:water:tetrahydrofuran at 30° C. (peak detection at 265 nm). The mean value from the 90 min dissolution test results of the six capsules was reported (Table 5).

TABLE 4
Chemical stability of calcitriol formulation in hard gelatin capsules
(225 mg total mass filled per capsule, 45 μg calcitriol)

| Storage | Time | Assay^{a} (%) | | | |
|---|---|---|---|---|---|
| Condition | (mos) | Form. 1 | Form. 2 | Form 3 | Form 4 |
| N/A | 0 | 100.1 | 98.8 | 99.1 | 100.3 |
| 5° C. | 1.0 | 99.4 | 98.9 | 98.9 | 104.3 |
| 25° C./60% RH | 0.5 | 99.4 | 97.7 | 97.8 | 102.3 |
| | 1.0 | 97.1 | 95.8 | 97.8 | 100.3 |
| | 3.0 | 95.2 | 93.6 | 96.8 | 97.9 |
| 30° C./65% RH | 0.5 | 98.7 | 97.7 | 96.8 | 100.7 |
| | 1.0 | 95.8 | 96.3 | 97.3 | 100.4 |
| | 3.0 | 94.2 | 93.6 | 95.5 | 93.4 |
| 40° C./75% RH | 0.5 | 96.4 | 96.7 | 98.2 | 97.1 |
| | 1.0 | 96.1 | 98.6 | 98.5 | 99.3 |
| | 3.0 | 92.3 | 92.4 | 93.0 | 96.4 |

^{a}Assay results indicate % of calcitriol relative to expected value based upon 45 μg content per capsule. Values include pre-calcitriol which is an active isomer of calcitriol.

TABLE 5
Physical Stability of Calcitriol Formulation in Hard Gelatin
Capsules (225 mg total mass filled per capsule, 45 μg calcitriol)

| Storage | Time | Dissolution^{a} (%) | | | |
|---|---|---|---|---|---|
| Condition | (mos) | Form. 1 | Form. 2 | Form 3 | Form 4 |
| N/A | 0 | 70.5 | 93.9 | 92.1 | 100.1 |
| 5° C. | 1.0 | 71.0 | 92.3 | 96.0 | 100.4 |
| 25° C./60% RH | 0.5 | 65.0 | 89.0 | 90.1 | 98.3 |
| | 1.0 | 66.1 | 90.8 | 94.5 | 96.2 |
| | 3.0 | 64.3 | 85.5 | 90.0 | 91.4 |
| 30° C./65% RH | 0.5 | 62.1 | 88.8 | 91.5 | 97.9 |
| | 1.0 | 65.1 | 89.4 | 95.5 | 98.1 |
| | 3.0 | 57.7 | 86.4 | 89.5 | 88.8 |
| 40° C./75% RH | 0.5 | 91.9 | 90.2 | 92.9 | 93.1 |
| | 1.0 | 63.4 | 93.8 | 94.5 | 95.2 |
| | 3.0 | 59.3 | 83.6 | 87.4 | 91.1 |

^{a}Dissolution of capsules was performed as described and the % calcitriol is calculated based upon a standard and the expected content of 45 μg calcitriol per capsule. The active isomer, pre-calcitriol, is not included in the calculation of % calcitriol dissolved. Values reported are from the 90 min sample.

The chemical stability results indicated that decreasing the MIGLYOL 812 content with a concomitant increase in Vitamin E TPGS content provided enhanced recovery of intact calcitriol as noted in Table 5. Formulation 4 (50:50 MIGLYOL 812/Vitamin E TPGS) was the most chemically stable formulation with only minor decreases in recovery of intact calcitriol after 3 months at 25° C./60% RH, enabling room temperature storage.

The physical stability of the formulations was assessed by the dissolution behavior of the capsules after storage at each stability condition. As with the chemical stability, decreasing the MIGLYOL 812 content and increasing the Vitamin E TPGS content improved the dissolution properties of the formulation (Table 6). Formulation 4 (50:50 MIGLYOL 812/Vitamin E TPGS) had the best dissolution properties with suitable stability for room temperature storage.

Having now fully described the invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method for treating or ameliorating lung cancer in an animal comprising administering to the animal a therapeutically effective amount of an active vitamin D compound or a mimic thereof by high dose pulse administration in combination with one or more chemotherapeutic agents or radiotherapeutic agents/treatments.

2. The method of claim 1, wherein said lung cancer is selected from the group consisting of non-small cell lung cancer, squamous cell carcinoma, epidermoid carcinoma, adenocarcinoma, bronchoalveolar cell carcinoma, small cell lung cancer, oat cell carcinoma, large-cell carcinoma, giant cell carcinoma, clear cell carcinoma, adenosquamous carcinoma, carcinoid tumor, bronchial gland carcinoma, soft-tissue tumor, and mesothelial tumor.

3. The method of claim 2, wherein said lung cancer is non-small cell lung cancer.

4. The method of claim 1, wherein said one or more chemotherapeutic agents is selected from the group consisting of cisplatin, carboplatin, paclitaxel, docetaxel, etoposide, vincristine, vinblastine, cyclophosphamide, doxorubicin, vinorelbine, topotecan, gemcitabine, irinotecan, gifitinib, ifosfamide, tarceva, oblimersen, TLK286, and any combination thereof.

5. The method of claim 4, wherein said one or more chemotherapeutic agents is docetaxel.

6. The method of claim 5, wherein said docetaxel is administered at a dose of 75 mg/m² body surface area.

7. The method of claim 1, wherein said one or more radiotherapeutic agents/treatments is selected from the group consisting of external-beam radiation therapy, brachytherapy, thermotherapy, radiosurgery, charged-particle radiotherapy, neutron radiotherapy, photodynamic therapy, radionuclide therapy, and any combination thereof.

8. The method of claim 1, wherein both one or more chemotherapeutic agents and one or more radiotherapeutic agents/treatments are administered.

9. The method of claim 1, wherein said active vitamin D compound or a mimic thereof is administered at least 12 hours prior to the administration of said one or more chemotherapeutic agents or radiotherapeutic agents/treatments.

10. The method of claim 9, wherein said active vitamin D compound or a mimic thereof is administered for 1 day to about 3 months prior to the administration of said one or more chemotherapeutic agents or radiotherapeutic agents/treatments.

11. The method of claim 1, wherein said active vitamin D compound or a mimic thereof is administered concurrently with the administration of said one or more chemotherapeutic agents or radiotherapeutic agents/treatments.

12. The method of claim 11, wherein the administration of said active vitamin D compound or a mimic thereof is continued beyond the administration of said one or more chemotherapeutic agents or radiotherapeutic agents/treatments.

13. The method of claim 1, wherein said active vitamin D compound or a mimic thereof is administered after the administration of said one or more chemotherapeutic agents or radiotherapeutic agents/treatments.

14. The method of claim 1, wherein the method is repeated at least once.

15. The method of claim 14, wherein said active vitamin D compound or a mimic thereof may be the same or different in each repetition and said one or more chemotherapeutic agents or radiotherapeutic agents/treatments may be the same or different in each repetition.

16. The method of claim 14, wherein the time period of administration of said active vitamin D compound or a mimic thereof may be the same or different in each repetition.

17. The method of claim 1, wherein said active vitamin D or a mimic thereof compound is calcitriol.

18. The method of claim 1, wherein said active vitamin D compound or a mimic thereof is 25-OH vitamin D₃.

19. The method of claim 1, wherein said active vitamin D compound or a mimic thereof has a reduced hypercalcemic effect.

20. The method of claim 19, wherein said active vitamin D compound or a mimic thereof is selected from the group consisting of EB 1089, Ro23-7553, and Ro24-5531.

21. The method of claim 1, wherein said active vitamin D compound or a mimic thereof is administered no more frequently than once in three days.

22. The method of claim 21, wherein said active vitamin D compound or a mimic thereof is administered no more frequently than once a week.

23. The method of claim 22, wherein said active vitamin D compound or a mimic thereof is administered no more frequently than once every three weeks.

24. The method of claim 1, wherein said active vitamin D compound or a mimic thereof is administered at a dose of about 15 μg to about 1 mg.

25. The method of claim 24, wherein said active vitamin D compound or a mimic thereof is administered at a dose of about 30 μg to about 300 μg.

26. The method of claim 25, wherein said active vitamin D compound or a mimic thereof is administered at a dose of about 165 μg.

27. The method of claim 1, wherein said active vitamin D compound or a mimic thereof is calcitriol and said one or more chemotherapeutic agents is docetaxel.

28. The method of claim 27, wherein said calcitriol is administered at a dose of 45 μg and said docetaxel is administered at a dose of 75 mg/m² body surface area.

29. The method of claim 27, wherein said calcitriol is administered at a dose of 75 μg and said docetaxel is administered at a dose of 75 mg/m² body surface area.

30. The method of claim 27, wherein said calcitriol is administered at a dose of 105 μg and said docetaxel is administered at a dose of 75 mg/m² body surface area.

31. The method of any one of claims 28-30, wherein said calcitriol is administered once every three weeks and said docetaxel is administered one day after each dose of calcitriol.

32. The method of claim 1, wherein said active vitamin D compound or a mimic thereof is administered orally, intravenously, parenterally, rectally, topically, nasally or transdermally.

33. The method of claim 31, wherein said active vitamin D compound or a mimic thereof is administered orally or intravenously.

34. The method of claim 1, further comprising reducing the level of calcium in the blood of the animal.

35. The method of claim 34, wherein said reducing comprises eating a reduced calcium diet, trapping calcium with an adsorbent, absorbent, ligand, chelate, or other calcium binding moiety that cannot be transported into the blood through the small intestine, administering a bisphosphonate or corticosteroid, increasing hydration and salt intake, or diuretic therapy.

36. The method of claim 1, wherein said administration is prior to surgery for resection of said lung cancer.

37. The method of claim 1, wherein said administration is after surgery for resection of said lung cancer.

38. The method of claim 1, wherein said active vitamin D compound is administered as a unit dosage form comprising about 50% MIGLYOL 812 and about 50% tocopherol PEG-1000 succinate (vitamin E TPGS).

39. The method of claim 38, wherein said unit dosage form further comprises at least one additive selected from the group consisting of an antioxidant, a bufferant, an antifoaming agent, a detackifier, a preservative, a chelating agent, a viscomodulator, a tonicifier, a flavorant, a colorant, an odorant, an opacifier, a suspending agent, a binder, a filler, a plasticizer, a thickening agent, a lubricant, and mixtures thereof.

40. The method of claim 39, wherein one of said additives is an antioxidant.

41. The method of claim 40, wherein said antioxidant is selected from the group consisting of butylated hydroxyanisole (BHA), butylated hydroxytoluene (BHT), and both.

42. The method of claim 41, wherein said unit dosage form comprises BHA and BHT.

43. The method of claim 42, wherein said unit dosage form comprises about 50% MIGLYOL 812, about 50% vitamin E TPGS, about 0.05% to about 0.35% BHA, and about 0.05% to about 0.35% BHT.

44. The method of claim 43, wherein said unit dosage form comprises about 50% MIGLYOL 812, about 50% vitamin E TPGS, about 0.35% BHA, and about 0.10% BHT.

45. The method of claim 38, wherein said unit dosage form is a capsule.

46. The method of claim 45, wherein said capsule is a gelatin capsule.

47. The method of claim 45, wherein the total volume of ingredients in said capsule is 10-1000 μl.

48. The method of claim 38, wherein said unit dosage form comprises about 10 μg to about 75 μg of calcitriol.

49. The method of claim 48, wherein said unit dosage form comprises about 45 μg of calcitriol.

50. The method of claim 49, wherein said unit dosage form comprises about 45 μg of calcitriol, about 50% MIGLYOL 812, about 50% vitamin E TPGS, BHA, and BHT.

51. The method of claim 49, wherein said unit dosage form comprises about 45 μg of calcitriol, about 50% MIGLYOL 812, about 50% vitamin E TPGS, about 0.35% BHA, and about 0.10% BHT.
